(19) United States
(12) Patent Application Publication
(10) Pub. No.: US 2024/0025868 A1
(43) Pub. Date: Jan. 25, 2024

(54) PHENOTHIAZINE COMPOUNDS FOR THE TREATMENT OF ALZHEIMER'S DISEASE AND OTHER AGE-RELATED AND NEUROLOGICAL DISEASES

(71) Applicant: Icahn School of Medicine at Mount Sinai, New York, NY (US)

(72) Inventors: Charles Mobbs, New York, NY (US); Jian Jin, New York, NY (US); Christopher Kellner, New York, NY (US); H. Umit Kaniskan, New York, NY (US); Tao Cao, New York, NY (US); Jianping Hu, New York, NY (US); James Vicari, New York, NY (US); Bik Tzu Huang, New York, NY (US); Yonejung Yoon, New York, NY (US); Rachel Litke, New York, NY (US)

(21) Appl. No.: 18/036,095

(22) PCT Filed: Nov. 12, 2021
(86) PCT No.: PCT/US2021/059156

§ 371 Date: May 9, 2023

Related U.S. Application Data
(60) Provisional application No. 63/113,683, filed on Nov. 13, 2020.

(51) Int. Cl. C07D 279/30; C07D 279/20; C07D 279/28; C07D 279/22; C07D 417/06; A61P 25/28; A61P 31/14

(52) U.S. Cl. CPC C07D 279/30; C07D 279/20; C07D 279/28; C07D 279/22; C07D 417/06; A61P 25/28; A61P 31/14

(57) ABSTRACT

Disclosed are phenothiazine compounds which reduce Abeta toxicity in vivo, reduce microglial inflammation as indicated by secretion of TNF-alpha, reduce intracerebral hemorrhage in vivo toxicity as indicated by preserved motor and cognitive function, and SARS-CoV-2 replication. Also disclosed are methods of use thereof for the treatment of disease in a subject in need thereof.

### STATEMENT REGARDING FEDERALLY SPONSORED RESEARCH

This invention was made with government support under AG062303 awarded by the National Institutes of Health. The government has certain rights in the invention.

### TECHNICAL FIELD

This disclosure relates to phenothiazine compounds which reduce Abeta toxicity in vivo, reduce microglial inflammation as indicated by secretion of TNF-alpha, reduce intracerebral hemorrhage in vivo toxicity as indicated by preserved motor and cognitive function, and SARS-CoV-2 replication. This disclosure also pertains to methods of use thereof for the treatment of disease in a subject in need thereof. The disclosure also relates to methods for designing such compounds.

### BACKGROUND OF THE INVENTION

Alzheimer's Disease (AD) constitutes one of the greatest threats to public health in the 21^{st} century. In the US alone the direct costs of AD are estimated to be $236 billion, with indirect financial costs to caregivers estimated to be almost as much (Alzheimer's 2016). The emotional toll on caregivers can also be overwhelming (Raggi, Tasca et al. 2015). Currently four individual drugs and one combination drug are approved for use in patients with AD (Greig 2015), but the effects of these drugs are relatively modest and the clinical significance of these effects, especially whether the benefits outweigh the harm, have been widely questioned (Peters 2013). For example, Eliti (Eleti 2016) simply states that “the efficacy of these is widely debated.” Clearly better pharmaceutical treatments for AD are of the utmost priority.

Age is by far the main risk factor for Alzheimer' s Disease (AD), which is associated with impaired cognitive function and is clinically defined by the accumulation of histological tangles and plaques, the main constituent of which is the Abeta 1-42 peptide (Wang and Ding 2008). Remarkably, muscle-specific transgenic expression of the human Abeta 1-42 peptide in C. elegans in the CL2006 strain (Link 2001) produces paralysis at about 80% of the average lifespan (Zhang, Poplawski et al. 2009), similar to the relative point in lifespan at which humans exhibit AD (de Pedro-Cuesta, Rabano et al. 2015). Similarly, transgenic expression of the human DNA sequence that causes Huntington's Disease produces similar motor impairments in mice and C. elegans at about the same relative age (i.e., at about 50% of average lifespan (Hodgson, Agopyan et al. 1999) of each species (de Pedro-Cuesta, Rabano et al. 2015) (Link 2001). Furthermore, dietary restriction (DR) increases lifespan in all animal species examined, and delays essentially all age-related diseases that have been examined in detail (Mobbs and Hof 2007), including in all models of AD that have been examined (Zhang, Poplawski et al. 2009).

A major challenge in the field is that drugs targeting Abeta itself may be effective in mouse models of AD but have failed in human AD trials (Mehta, Jackson et al. 2017), leading to proposals that multi-functional drugs or combinations will be necessary to treat AD (Luo, Lee et al. 2016). Proteotoxicity is a common mechanism driving neurodegenerative diseases and human AD is characterized by at least two forms of proteotoxicity defined by both plaques (entailing Abeta aggregation) and tangles (entailing pTau aggregation) (Spires-Jones, Attems et al. 2017). Therefore we hypothesize that interventions that reduce in vivo toxicity produced by the Abeta peptide, without targeting Abeta itself, are likely to be most effective against AD.

Logistically, C. elegans offers many advantages for carrying out such screens, and indeed is by far the main organism in which such high-throughput phenotypic screens for drugs (Evason, Huang et al. 2005) (Petrascheck, Ye et al. 2007) (Ye, Linton et al. 2014, Lucanic, Garrett et al. 2016) have been carried out. For example, we discovered a plausible treatment for AD by demonstrating that HDAC inhibitor sodium butyrate significantly reduces Abeta toxicity in vivo in a C. elegans model of AD (Zhang, Poplawski et al. 2009). These results were corroborated and extended by studies demonstrating the protective effects of sodium butyrate in a mouse neuron-specific double-transgenic (A-beta/presenilin) model of AD (Govindarajan, Agis-Balboa et al. 2011). In this mouse model Abeta plaques and cognitive impairments develop by 15 months of age. This study demonstrated treatment with sodium butyrate between 14 and 15 months reversed cognitive impairments and reduced acetylation of H3K14, H4K5, and H4K16 with no changes in A-beta plaques. A significant implication of this observation is that, the protective effect of HDAC inhibition on Abeta toxicity is mediated by processes subsequent to A-beta production and aggregation, targeting of which has failed in clinical trials (Mehta, Jackson et al. 2017).

While Abeta and tau proteotoxicity are hallmark features of Alzheimer's Disease, genetic (GWAS) studies have increasingly implicated microglial activity, particularly excessive inflammation, as driving pathology in the disease (Kaur, Sharma et al. 2019). An essential component of the inflammatory process in the brain is the secretion of the cytokine TNF-alpha, which extensive research has independently implicated in driving pathological processes in Alzheimer's Disease (Decourt, Lahiri et al. 2017), including associated psychosis (Holmes, Cunningham et al. 2011). Inflammation is causally implicated in driving pathophysiological processes many, possibly all, age-related diseases (Franceschi, Bonafe et al. 2000). For example, Humira, an antibody which specifically targets and inhibits TNF-alpha, has long been the best-selling drug in the world by virtue of its beneficial effects on a wide range of age-related diseases (Ladika 2019), although as an antibody Humira does not cross the blood-brain barrier. Nevertheless TNF-alpha is similarly implicated in many, possibly all, neurological diseases in addition to Alzheimer's Disease, Parkinson's Disease (Dobbs, Charlett et al. 1999), Huntington's Disease (Yang, Yang et al. 2017), intracerebral hemorrhage (ICH) (Ma, Jiang et al. 2018), ischemic stroke (Pawluk, Wozniak et al. 2020), traumatic brain injury (Cheong, Chang et al. 2013, Chio, Chang et al. 2013), and even eye disease including glaucoma (Yang, Zeng et al. 2020). In addition, plasma TNF-alpha is currently the best indicator of outcome in COVID-19, whose mortality rate exponentially increases with age (Del Valle, Kim-Schulze et al. 2020).

### SUMMARY OF THE INVENTION

Unlike previous compounds developed to treat Alzheimer' s Disease and other age-related diseases, including mortality in COVID-19, which were largely unsuccessfully directed toward the presumptive distal cause of the disease, the novel phenothiazone compounds described herein were discovered based on screening of phenotypes particularly sensitive to the age-dependency of these diseases. The efficacy of these compounds to reduce toxicity are assessed, as reflected by age-dependent paralysis, in an Abeta transgenic C. elegans model of Alzheimer's Disease. Because microglial inflammation, especially the cytokine TNF-alpha, are implicated in driving the pathophysiology of Alzheimer's Disease, the efficacy of these compounds to inhibit microglial secretion of TNF-alpha, in response to Abeta peptide, was also assessed.

### Compounds

Compounds according to the invention include compounds according to one of the following Formulas:

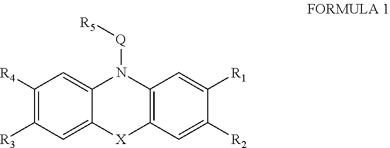

wherein
X is selected from S or S═O;
Q is selected from CH₂ or CO;
R₁, R₂, R₃ and R₄, are selected from hydrogen, halogen, F, Br, Cl, CH₃, CF₃, OCH₃, COCH₃, CN, C(O)R⁸, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,

Wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; and

R₅ is selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl;

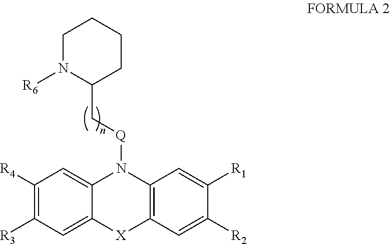

wherein

X is selected from S or S═O;
Q is selected from CH₂ or CO;
n is selected from 0, 1, 2 or 3;
the definitions of R¹, R² , R³ , and R⁴ are the same as for FORMULA 1;
R⁶ is selected from hydrogen, CH₃, CF₃, OCH₃, COCH₃, CN, C(O)R⁸, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃ -C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,

Wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl;

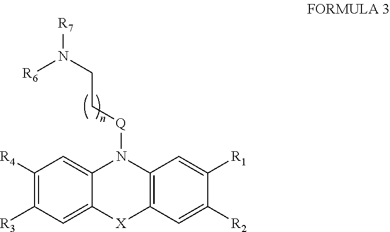

wherein
X is selected from S or S═O;
Q is selected from CH₂ or CO;
n is selected from 0, 1, 2 or 3;
the definitions of R¹, R², R³, R⁴ and R⁶ are the same as for FORMULA 2; and

R⁷ is selected from hydrogen, CH₃, CF₃, OCH₃, COCH₃, CN, C(O)R⁸ , optionally substituted C₁-C₈ alkyl, optionally substituted C₁-C₈alkoxyC₁-C₈alkyl, optionally substituted C₁-C₈alkylaminoC₁-C₈alkyl, optionally substituted C₃ -C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,

Wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl;

and pharmaceutically acceptable salts and enantiomers thereof.

In some aspects, a compound according to the present invention is a compound selected from those synthesized in the Examples below, including, but not limited to: TC₈₄-31, TC₈₄-37, TC₈₄-32, TC84-38, TC84-13, TC84-20, TC84-39, TC84-110, TC84-144, TC84-125, TC96-20, TC84-124, TC₈₄-161, TC₈₄-162, TC₈₄-163, TC₉₆-32 and pharmaceutically acceptable salts and enantiomers thereof, and analogs thereof.

In some aspects of the disclosed methods, the compounds can be administered by any of several routes of administration including, e.g., orally, parenterally, intradermally, subcutaneously, topically, and/or rectally.

Any of the above-described methods can further include treating the subject with one or more additional therapeutic regimens for treatment.

As used herein, the terms “about” and “approximately” are defined as being within plus or minus 10% of a given value or state, preferably within plus or minus 5% of said value or state. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1. Phenothiazines preserve motor and cognitive function in a mouse model of intracerebral hemorrhage. Compounds #45 (Chlorprothixen) #75 (Triflupromazine) and #310 (example 8/TC₈₄-110) preserve motor and cognitive function compared to DMSO. Motor function was assessed using rotarod and and cognitive was assessed by alternation in a Y-maze. All data were normalized to data pre-ICH function.

FIG. 2. Plasma and brain concentrations of TC₀₈₄-110 over 8 h following a single IV injection (A) at 1 mg/kg or oral administration (B) at 10 mg/kg in male C₅₇BL/6 mice. TC₀₈₄-110 has very high brain/plasma ratio and is orally available. Plasma and brain concentrations at each time point represent the mean+/−SD from 3 mice.

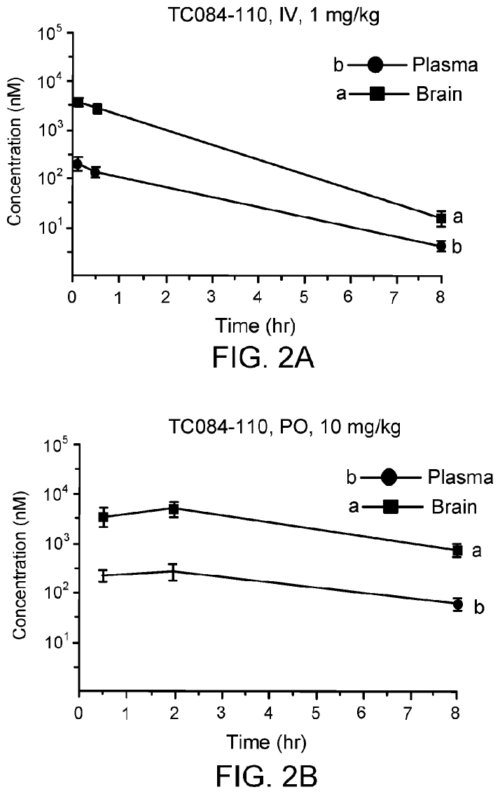

### DETAILED DESCRIPTION

Compounds according to the invention include compounds according to one of the following Formulas:

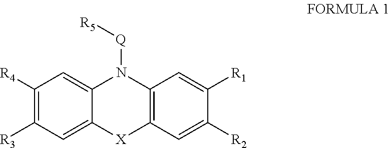

wherein
X is selected from S or S═O;
Q is selected from CH₂ or CO;
R₁, R₂, R₃ and R₄, are selected from hydrogen, halogen, F, Br, Cl, CH₃, CF₃, OCH₃, COCH₃, CN, C(O)R⁸ , optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,

Wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl;

R₅ is selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl;

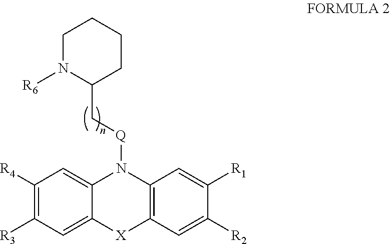

wherein
X is selected from S or S═O;
Q is selected from CH₂ or CO;
n is selected from 0, 1, 2 or 3;
the definitions of R¹, R², R³, and R⁴ are the same as for FORMULA 1;
R₆ is selected from hydrogen, CH₃, CF₃, OCH₃, COCH₃, CN, C(O)R⁸, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃ -C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,

Wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; and

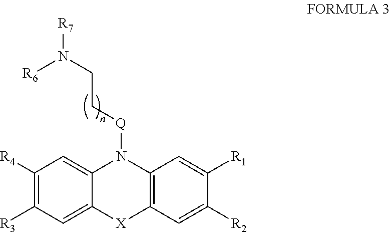

wherein
X is selected from S or S═O;
Q is selected from CH₂ or CO;
n is selected from 0, 1, 2 or 3;
the definitions of R¹, R², R³, R⁴ and R⁶ are the same as for FORMULA 2;
R₇ is selected from hydrogen, CH₃, CF₃, OCH₃, COCH₃, CN, C(O)R⁸, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃ -C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,

Wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; and

and pharmaceutically acceptable salts and enantiomers thereof.

In some aspects, a compound according to the present invention is a compound selected from those synthesized in the Examples below, including, but not limited to: TC₈₄-31, TC₈₄-37, TC₈₄-32, TC84-38, TC84-13, TC84-20, TC84-39, TC84-110, TC84-144, TC84-125, TC96-20, TC84-124, TC₈₄-161, TC₈₄-162, TC₈₄-163, TC₉₆-32 and pharmaceutically acceptable salts and enantiomers thereof and analogs thereof.

In some aspects of the disclosed methods, the compounds can be administered by any of several routes of administration including, e.g., orally, parenterally, intradermally, subcutaneously, topically, and/or rectally.

Any of the above-described methods can further include treating the subject with one or more additional therapeutic regimens for treatment.

As used herein, the terms “about” and “approximately” are defined as being within plus or minus 10% of a given value or state, preferably within plus or minus 5% of said value or state.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### Synthesis and Testing of the Compounds

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

### Definition of Terms

As used herein, the terms “comprising” and “including” are used in their open, non-limiting sense.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl).

In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1 -enyl, pent-1 -enyl, penta-1,4-dienyl, and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein witch is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, ″ refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group. The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclyl group may include fused, bridged or spirocyclic ring systems. In certain embodiments, a hetercyclyl group comprises 3 to 10 ring atoms (3-10 membered heterocyclyl). In certain embodiments, a hetercyclyl group comprises 3 to 8 ring atoms (3-8 membered heterocyclyl). In certain embodiments, a hetercyclyl group comprises 4 to 8 ring atoms (4-8 membered heterocyclyl). In certain embodiments, a hetercyclyl group comprises 3 to 6 ring atoms (3-6 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0] hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “ cycloalkyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (C₃-C₈ cycloalkyl). In certain embodiments, a cycloalkyl comprises 3 to 6 carbon ring atoms (C₃-C₆ cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro [3.4]octane, and spiro [4.5]decane.

The term cyano” refers to a —C≡T group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)—group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, -C₁-C₄ alkylphenyl, alkyl-OH, haloalkyl, halo, —OH, —NH₂, -C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Pharmaceutical Compositions

In some aspects, the compositions and methods described herein include the manufacture and use of pharmaceutical compositions and medicaments that include one or more compounds as disclosed herein. Also included are the pharmaceutical compositions themselves.

In some aspects, the compositions disclosed herein can include other compounds, drugs, or agents used for the treatment. For example, in some instances, pharmaceutical compositions disclosed herein can be combined with one or more (e.g., one, two, three, four, five, or less than ten) compounds.

In some aspects, the pH of the compositions disclosed herein can be adjusted with pharmaceutically acceptable acids, bases, or buffers to enhance the stability of the compounds or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivative thereof.

In particular, pharmaceutically acceptable salts of the compounds disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., alkali metal (e.g., sodium), alkaline earth metal (e.g., magnesium), ammonium salts. The invention also envisions the quaternization of any basic nitrogen-containing groups of the compounds disclosed herein. Water or oil-soluble or dispersible products can be obtained by such quaternization.

In some aspects, the pharmaceutical compositions disclosed herein can include an effective amount of one or more compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome. In some aspects, pharmaceutical compositions can further include one or more additional compounds, drugs, or agents used for the treatment in amounts effective for causing an intended effect or physiological outcome.

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added. For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein and one or more additional compounds (e.g., one or more additional compounds, drugs, or agents used for the treatment of Alzeimers Disease (AD) or any other age related condition or disease, including conditions or diseases known to be associated with or caused by AD), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention.

Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising a compound, including pharmaceutical compositions (indicated below as ‘X’) disclosed herein in the following methods: Substance X for use as a medicament in the treatment of one or more diseases or conditions disclosed herein. Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of AD or age related diseases.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering. In some aspects, methods of treatment can include assessing a level of disease in the subject prior to treatment, during treatment, or after treatment. In some aspects, treatment can continue until a decrease in the level of disease in the subject is detected. The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder are ameliorated or otherwise beneficially altered. As used herein, amelioration of the symptoms of a particular disorder refers to any lessening, whether permanent or temporary, lasting or transient that can be attributed to or associated with treatment by the compositions and methods of the present invention. In some aspects, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. As used herein, the term “preventing a disease” in a subject means for example, to stop the development of one or more symptoms of a disease in a subject before they occur or are detectable, e.g., by the patient or the patient's doctor. Preferably, the disease does not develop at all, i.e., no symptoms of the disease are detectable. However, it can also mean delaying or slowing of the development of one or more symptoms of the disease. Alternatively, or in addition, it can mean decreasing the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

### EXAMPLES

The following Examples describe the synthesis of exemplary compounds according to the present invention.

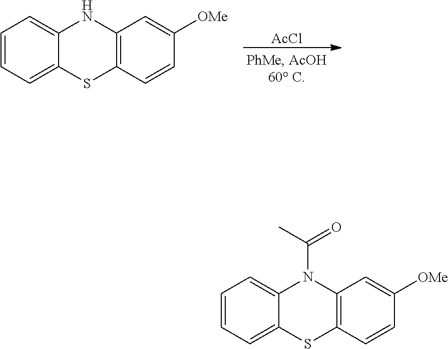 [SMILES: "AcCl" ; COc1ccc2c(c1)Nc1ccccc1S2 ; COc1ccc2c(c1)N(C(C)=O)c1ccccc1S2]

Example 1:1-(2-methoxy-10H-phenothiazin-10-yl)ethan-1-one (TC₈₄-31): To the suspension of commercially available 2-methoxy-10H-phenothiazine (229.3 mg, 1 mmol) in toluene (5 mL) and acetic acid (5 mL) was added acetyl chloride (0.7 mL, 10 mmol). After being stirred for 30 min at 60 ° C., the resulting mixture was concentrated and purified by silica gel flash chromatography (30-50% ethyl acetate in hexane) to give the title compound as oil (271.2 mg, 100%). ¹H NMR (600 MHz, Chloroform-d) δ 7.53-7.42 (m, 2H), 7.36-7.29 (m, 2H), 7.25-7.20 (m, 1H), 7.11 (s, 1H), 6.85-6.82 (m, 1H), 3.83 (s, 3H), 2.24 (s, 3H). HRMS m/z [M+H]⁺ calcd for C₁₅H₁₄NO₂S⁺ 272.0740, found 272.0731.

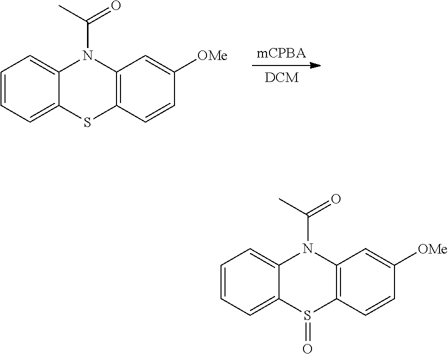 [SMILES: COc1ccc2c(c1)N(C(C)=O)c1ccccc1S2 ; COc1ccc2c(c1)N(C(C)=O)c1ccccc1S2=O]

Example 2: 1-(2-methoxy-5-oxido-10H-phenothiazin-10-yl)ethan-1-one (TC₈₄-37): To the solution of 1-(2-methoxy-10H-phenothiazin-10-yl)ethan-1-one (54.3 mg, 0.2 mmol) in DCM (4 mL) was added mCPBA (46.0 mg, 75% purity, 0.2 mmol). After being stirred for 1 h at room temperature, the resulting mixture was concentrated and purified by preparative HPLC (10-100% methanol/0.1% TFA in H₂O) to give the title compound as oil (47.2 mg, 81%). ¹H NMR (600 MHz, Methanol-d₄) δ 7.86 (t, J=6.0 Hz, 2H), 7.77 (d, J=8.6 Hz, 1H), 7.63 (t, J=7.7 Hz, 1H), 7.52 (t, J=7.5 Hz, 1H), 7.45 (d, J=2.4 Hz, 1H), 7.08 (dd, J=8.7, 2.4 Hz, 1H), 3.90 (s, 3H), 2.37 (s, 3H). HRMS m/z [M+H]⁺ calcd for C₁₅H₁₄NO₃S⁺ 288.0689, found 288.0692.

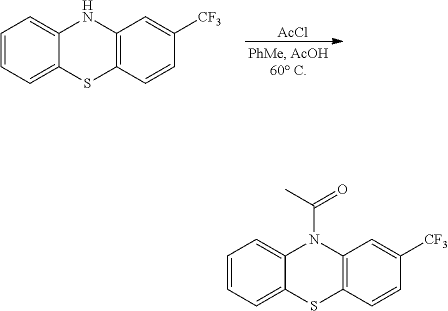 [SMILES: "AcCl" ; FC(F)(F)c1ccc2c(c1)Nc1ccccc1S2 ; CC(=O)N1c2ccccc2Sc2ccc(C(F)(F)F)cc21]

Example 3: 1-(2-(trifluoromethyl)-10H-phenothiazin-10-yl)ethan-1-one (TC₈₄-32): To the suspension of commercially available 2-(trifluoromethyl)-10H-phenothiazine (267.3 mg, 1 mmol) in toluene (5 mL) and acetic acid (5 mL) was added acetyl chloride (0.7 mL, 10 mmol). After being stirred for 30 min at 60° C., the resulting mixture was concentrated and purified by silica gel flash chromatography (15-30% ethyl acetate in hexane) to give the title compound as oil (308.9 mg, 100%). ¹H NMR (600 MHz, Chloroform-d) δ 7.84 (s, 1H), 7.59-7.26 (m, 6H), 2.25 (s, 3H). HRMS m/z [M+H]⁺ calcd for C₁₅H₁₁F₃NOS⁺ 310.0508, found 310.0511.

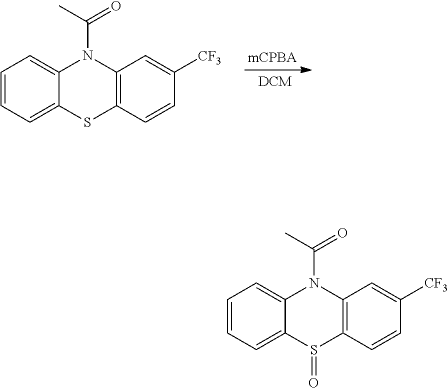 [SMILES: CC(=O)N1c2ccccc2Sc2ccc(C(F)(F)F)cc21 ; CC(=O)N1c2ccccc2S(=O)c2ccc(C(F)(F)F)cc21]

Example 4: 1-(5-oxido-2-(trifluoromethyl)-10H-phenothiazin-10-yl)ethan-1-one (TC₈₄-38): To the solution of 1-(2-(trifluoromethyl)-10H-phenothiazin-10-yl)ethan-1-one (61.8 mg, 0.2 mmol) in DCM (4 mL) was added mCPBA (46.0 mg, 75% purity, 0.2 mmol). After being stirred for 1 h at room temperature, the resulting mixture was concentrated and purified by preparative HPLC (10-100% methanol/0.1% TFA in H₂O) to give the title compound as oil (38.3 mg, 59%). ¹H NMR (600 MHz, Methanol-d₄) δ 8.24 (s, 1H), 8.05 (d, J=8.2 Hz, 1H), 7.91-7.85 (m, 3H), 7.68 (t, J=7.7 Hz, 1H), 7.62 (t, J=7.6, Hz, 1H), 2.39 (s, 3H). HRMS m/z [M+H]⁺ calcd for C₁₅H₁F₃NO₂S⁺ 326.0457, found 326.0462.

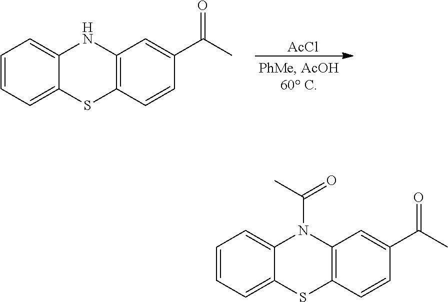 [SMILES: "AcCl" ; CC(=O)c1ccc2c(c1)Nc1ccccc1S2 ; CC(=O)c1ccc2c(c1)N(C(C)=O)c1ccccc1S2]

Example 5: 1,1′-(10H-phenothiazine-2,10-diyl)bis(ethan-1-one) (TC₈₄-13): To the suspension of commercially available 1-(10H-phenothiazin-2-yl)ethan-1-one (241.3 mg, 1 mmol) in toluene (5 mL) and acetic acid (5 mL) was added acetyl chloride (0.7 mL, 10 mmol). After being stirred for 30 min at 60° C., the resulting mixture was concentrated and purified by silica gel flash chromatography (15-50% ethyl acetate in hexane) to give the title compound as oil (244.2 mg, 87%). ¹H NMR (500 MHz, Chloroform-d) δ 8.12 (s, 1H), 7.84 (dd, J =8.2, 1.8 Hz, 1H), 7.60-7.45 (m, 3H), 7.39 (td, J=7.7, 1.5 Hz, 1H), 7.33-7.26 (m, 1H), 2.64 (s, 3H), 2.24 (s, 3H). HRMS m/z [M+H]⁺ calcd for C₁₆H₁₄NO₂S⁺ 284.0740, found 284.0735.

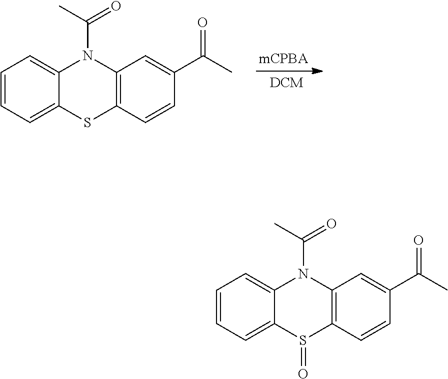 [SMILES: CC(=O)c1ccc2c(c1)N(C(C)=O)c1ccccc1S2 ; CC(=O)c1ccc2c(c1)N(C(C)=O)c1ccccc1S2=O]

Example 6: 1,1′-(5-oxido-10H-phenothiazine-2,10-diyl)bis(ethan-1-one) (TC₈₄-20): To the solution of 1,1′-(10H-phenothiazine-2,10-diyl)bis(ethan-1-one) (56.6 mg, 0.2 mmol) in DCM (4 mL) was added mCPBA (46.0 mg, 75% purity, 0.2 mmol). After being stirred for 1 h at room temperature, the resulting mixture was concentrated and purified by preparative HPLC (10-100% methanol/0.1% TFA in H₂O) to give the title compound as oil (50.8 mg, 85%). ¹H NMR (500 MHz, Methanol-d₄) δ 8.41 (s, 1H), 8.13 (d, J=8.1 Hz, 1H), 7.96 (d, J=8.1 Hz, 1H), 7.90-7.83 (m, 2H), 7.69-7.54 (m, 2H), 2.67 (s, 3H), 2.38 (s, 3H). HRMS m/z [M+H]⁺ calcd for C₁₆H₁₄NO₃S⁺ 300.0689, found 300.0682.

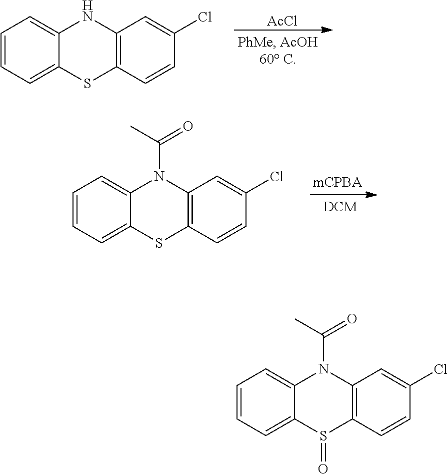

### Example 7

1-(2-chloro-5-oxido-10H-phenothiazin-10-yl)ethan-1-one (TC₈₄-39): To the suspension of commercially available 2-chloro-10H-phenothiazine (233.7 mg, 1 mmol) in toluene (5 mL) and acetic acid (5 mL) was added acetyl chloride (0.7 mL, 10 mmol). After being stirred for 30 min at 60° C., the resulting mixture was concentrated and purified by silica gel flash chromatography (15% ethyl acetate in hexane) to give 1-(2-chloro-10H-phenothiazin-10-yl)ethan-1-one. 1-(2-chloro-10H-phenothiazin-10-yl)ethan-1-one (55.1 mg, 0.2 mmol) in DCM (4 mL) was then added mCPBA (46.0 mg, 75% purity, 0.2 mmol). After being stirred for 1 h at room temperature, the resulting mixture was concentrated and purified by preparative HPLC (10-100% methanol/0.1% TFA in H₂O) to give the title compound as oil (45.6 mg, 77%). ¹H NMR (600 MHz, Methanol-d₄) δ 7.94 (d, J=2.0 Hz, 1H), 7.90 — 7.83 (m, 2H), 7.81 (d, J=8.4 Hz, 1H), 7.65 (td, J=7.7, 1.5 Hz, 1H), 7.60-7.52 (m, 2H), 2.36 (s, 3H). HRMS m/z [M+H]⁺ calcd for C₁₄H₁₁ClNO₂S⁺ 292.0194, found 292.0190.

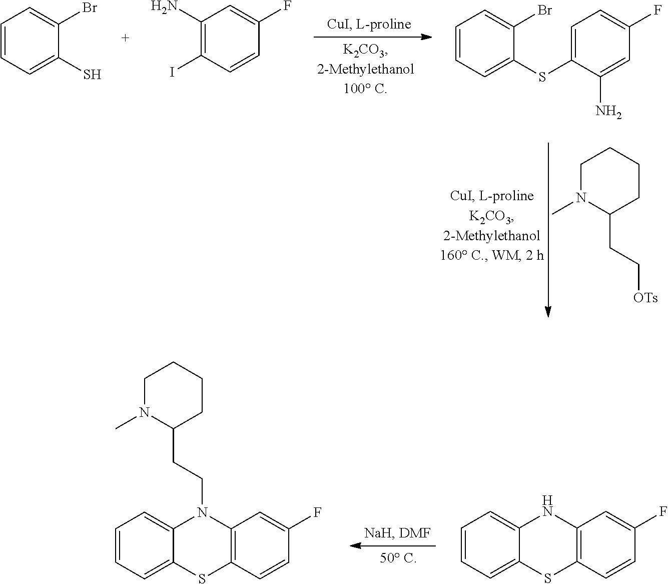

Example 8: 2-fluoro-10-(2-(1-methylpiperidin-2-yl)ethyl)-10H-phenothiazine (TC₈₄-110): To the suspension of CuI (190.5 mg, 1 mmol), L-proline (230.3 mg, 2 mmol), 5-fluoro-2-iodoaniline (1.18 g, 5 mmol), and K₂CO₃ (3.45 g, 25 mmol) in 2-methylethanol (10 mL) was added 2-bromobenzenethiol (0.65 mL, 5.5 mmol), under nitrogen atmosphere. After being stirred for 24 h at 50° C., the mixture was concentrated and purified by silica gel flash chromatography (10% ethyl acetate in hexane) to give 2-((2-bromophenyl)thio)-5-fluoroaniline. To the suspension of CuI (19.0 mg, 0.1 mmol), L-proline (23.0 mg, 0.2 mmol), and K₂CO₃ (345.5 mg, 2.5 mmol) in 2-methylethanol (5 mL) in a microwave tube was added 2-((2-bromophenyl)thio)-5-fluoroaniline (149.1 mg, 0.5 mmol). After being stirred for 2 h at 160° C. under microwave irradiation, the mixture was concentrated and purified by silica gel flash chromatography (10-50% ethyl acetate in hexane) to give 2-fluoro-10H-phenothiazine as solid (62.4 mg, 58%).

To the suspension of 2-fluoro-10H-phenothiazine (62.4 mg, 0.3 mmol) and sodium hydride (40 mg, 60% in mineral oil, 1 mmol) in DMF (2 mL) was added 2-(1-methylpiperidin-2-yl)ethyl 4-methylbenzenesulfonate (178.4 mg, 0.6 mmol). After being stirred for 1 h at 50° C., water was added, and the mixture was extracted with ethyl acetate (5 mL×3), dried over Na₂SO₄, filtered, and concentrated. The resulting mixture was purified by preparative HPLC (10-100% methanol/0.1% TFA in H₂O) to give the title compound as oil in its TFA salt form (45.2 mg, 34%). ¹H NMR (600 MHz, Methanol-d₄) δ 7.26 (t, J=7.5 Hz, 1H), 7.23-7.12 (m, 2H), 7.07 (d, J=8.2 Hz, 1H), 7.04-7.00 (m, 1H), 6.87 (dd, J=10.9, 2.5 Hz, 1H), 6.75 (tt, J=8.3, 2.5 Hz, 1H), 4.15-4.08 (m, 1H), 4.07-3.99 (m, 1H), 3.57-3.43 (m, 1H), 3.22-3.05 (m, 1H), 2.95 (td, J=12.7, 3.3 Hz, 1H), 2.70 (s, 2H), 2.65 (s, 1H), 2.56-2.49 (m, 0.7H), 2.23-2.17 (m, 0.3H), 2.09-1.89 (m, 2H), 1.88-1.66 (m, 3H), 1.61-1.42 (m, 2H). HRMS m/z [M+H]⁺ calcd for C₂₀H₂₄FN₂S⁺ 343.1639, found 343.1644.

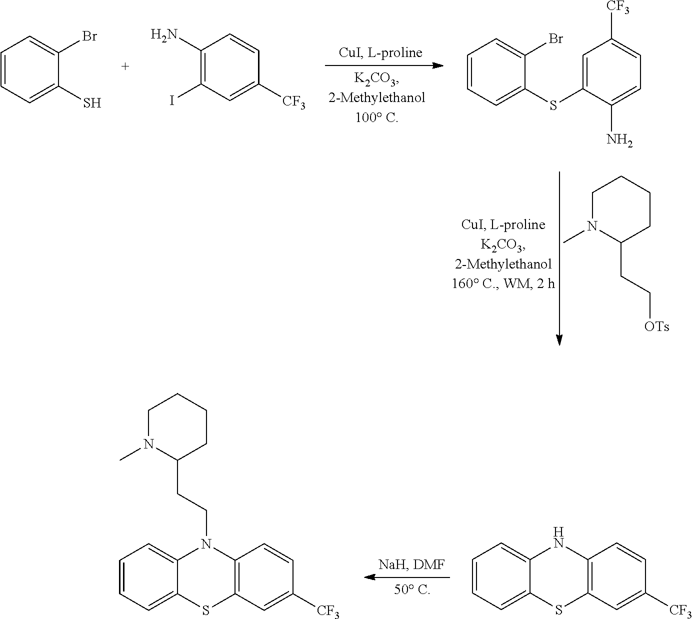

Example 9: 10-(2-(1-methylpiperidin-2-yl)ethyl)-3-(trifluoromethyl)-10H-phenothiazine (TC₈₄-144): To the suspension of CuI (190.5 mg, 1 mmol), L-proline (230.3 mg, 2 mmol), 2-iodo-4-(trifluoromethyl)aniline (1.43 g, 5 mmol), and K₂CO₃ (3.45 g, 25 mmol) in 2-methylethanol (10 mL) was added 2-bromobenzenethiol (0.65 mL, 5.5 mmol), under nitrogen atmosphere. After being stirred for 24 h at 50° C., the mixture was concentrated and purified by silica gel flash chromatography (10% ethyl acetate in hexane) to give 2-((2-bromophenyl)thio)-4-(trifluoromethyl)aniline (1.31 g, 75%). To the suspension of CuI (38.1 mg, 0.2 mmol), L-proline (46.1 mg, 0.4 mmol), and K₂CO₃ (691.1 mg, 5 mmol) in 2-methylethanol (5 mL) in a microwave tube was added 2-((2-bromophenyl)thio)-4-(trifluoromethyl)aniline (348.2 mg, 1 mmol). After being stirred for 2 h at 160° C. under microwave irradiation, the mixture was concentrated and purified by silica gel flash chromatography (10-50% ethyl acetate in hexane) to give 3-(trifluoromethyl)-10H-phenothiazine as solid.

To the suspension of 3-(trifluoromethyl)-10H-phenothiazine (53.5 mg, 0.2 mmol) and sodium hydride (24 mg, 60% in mineral oil, 0.6 mmol) in DMF (2 mL) was added 2-(1-methylpiperidin-2-yl)ethyl 4-methylbenzenesulfonate (119 mg, 0.4 mmol). After being stirred for 1 h at 50° C., water was added, and the mixture was extracted with ethyl acetate (5 mL×3), dried over Na₂SO₄, filtered, and concentrated. The resulting mixture was purified by preparative HPLC (10-100% methanol/0.1% TFA in H₂O) to give the title compound as oil in its TFA salt form (46.3 mg, 46%). ¹H NMR (600 MHz, Methanol-d 4) δ 7.56 (d, J=8.4 Hz, 1H), 7.49 (s, 1H), 7.32 (td, J=7.9, 1.5 Hz, 1H), 7.28-7.18 (m, 2H), 7.16-7.11 (m, 1H), 7.08 (t, J=7.5 Hz, 1H), 4.26-4.19 (m, 1H), 4.18-4.10 (m, 1H), 3.68 (s, 1H), 3.60-3.45 (m, 1H), 3.24-3.10 (m, 1H), 3.05-2.98 (m, 1H), 2.74 (s, 2H), 2.69 (s, 1H), 2.59-2.51 (m, 0.7H), 2.24-2.18 (m, 0.3H), 2.14-2.09 (m, 1H), 1.97-1.81 (m, 2H), 1.78-1.66 (m, 1H), 1.60-1.47 (m, 2H). HRMS m/z [M+H]⁺ calcd for C₂₁H₂₄F₃N₂S⁺ 393.1607, found 393.1606.

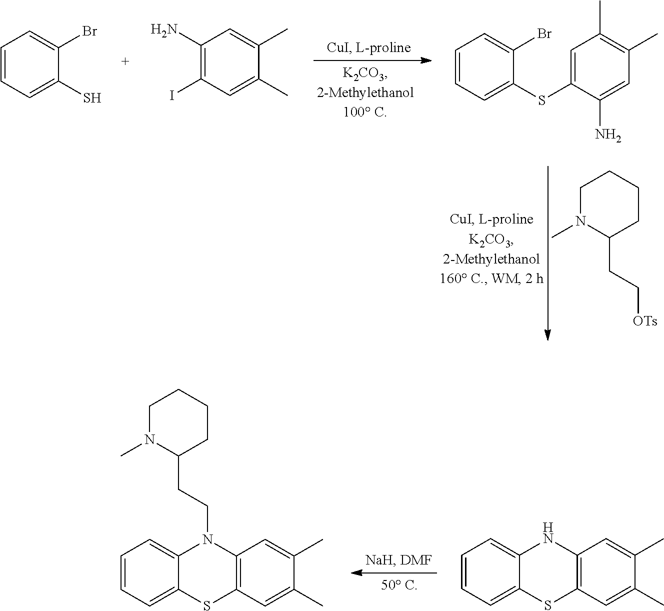

Example 10: 2,3-dimethyl-10-(2-(1-methylpiperidin-2-yl)ethyl)-10H-phenothiazine (TC₈₄-125): To the suspension of CuI (190.5 mg, 1 mmol), L-proline (230.3 mg, 2 mmol), 2-iodo-4,5-dimethylaniline (1.23 g, 5 mmol), and K₂CO₃ (3.45 g, 25 mmol) in 2-methylethanol (10 mL) was added 2-bromobenzenethiol (0.65 mL, 5.5 mmol) under nitrogen atmosphere. After being stirred for 24 h at 50° C., the mixture was concentrated and purified by silica gel flash chromatography (10% ethyl acetate in hexane) to give 2-((2-bromophenyl)thio)-4,5-dimethylaniline (1.28 g, 83%). To the suspension of CuI (38.1 mg, 0.2 mmol), L-proline (46.1 mg, 0.4 mmol), and K₂CO₃ (691.1 mg, 5 mmol) in 2-methylethanol (5 mL) in a microwave tube was added 2-((2-bromophenyl)thio)-4,5-dimethylaniline (308.2 mg, 1 mmol). After being stirred for 2 h at 160° C. under microwave irradiation, the mixture was concentrated and purified by silica gel flash chromatography (10-50% ethyl acetate in hexane) to give 2,3-dimethyl-10H-phenothiazine as a solid.

To the suspension of 2,3-dimethyl-10H-phenothiazine (22.7 mg, 0.1 mmol) and sodium hydride (24 mg, 60% in mineral oil, 0.6 mmol) in DMF (2 mL) was added 2-(1-methylpiperidin-2-yl)ethyl 4-methylbenzenesulfonate (60 mg, 0.2 mmol). After being stirred for 1 h at 50° C., water was added, and the mixture was extracted with ethyl acetate (5 mL×3), dried over Na₂SO₄, filtered, and concentrated. The resulting mixture was purified by preparative HPLC (10-100% methanol/0.1% TFA in H₂O) to give the title compound as oil in its TFA salt form (27.0 mg, 58%). ¹H NMR (600 MHz, Methanol-d₄) δ 7.23 (t, J=7.6 Hz, 1H), 7.18 (t, J=7.4 Hz, 1H), 7.03 (s, 1H), 6.99-6.95 (m, 1H), 6.93 (d, J=4.7 Hz, 1H), 6.83 (s, 1H), 4.15-4.08 (m, 1H), 4.05-3.98 (m, 1H), 3.57-3.40 (m, 1H), 3.19-3.04 (m, 1H), 2.91 (td, J=12.7, 3.3 Hz, 1H), 2.65 (s, 2H), 2.61 (s, 1H), 2.55-2.49 (m, 1H), 2.25 (s, 3H), 2.17 (s, 3H), 2.07-1.97 (m, 2H), 1.92-1.65 (m, 3H), 1.57-1.39 (m, 2H). HRMS m/z [M+H]⁺ calcd for C₂₂H₂₉N₂S⁺ 353.2046, found 353.2036.

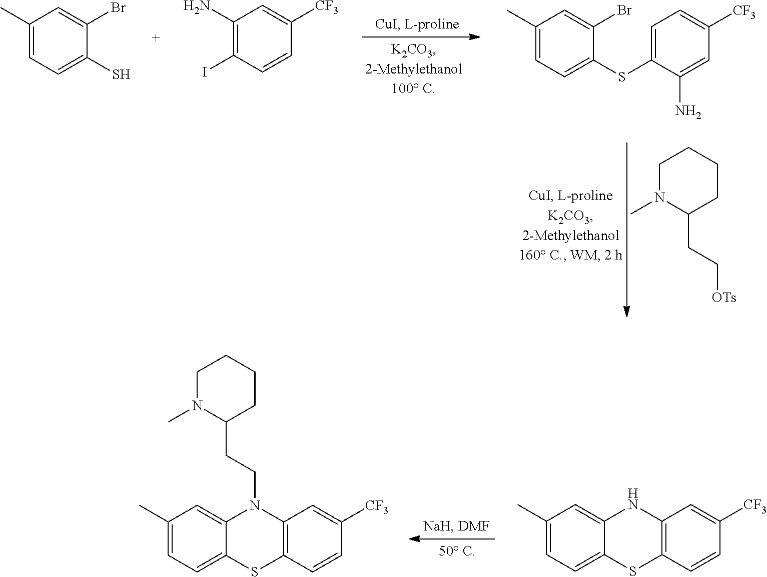

Example 11: 2-methyl-10-(2-(1-methylpiperidin-2-yl)ethyl)-8-(trifluoromethyl)-10H-phenothiazine (TC₉₆-20): To the suspension of CuI (38.1 mg, 0.2 mmol), L-proline (46.1 mg, 0.4 mmol), 2-iodo-5-(trifluoromethyl)aniline (0.15 mL, 1 mmol), and K₂CO₃ (691.1 mg, 5 mmol) in 2-methylethanol (10 mL) was added 2-bromo-4-methylbenzenethiol (0.2 mL, 1.1 mmol) under nitrogen atmosphere. After being stirred for 24 h at 50° C., the mixture was concentrated and purified by silica gel flash chromatography (10% ethyl acetate in hexane) to give 2-((2-bromo-4-methylphenyl)thio)-5-(trifluoromethyl)aniline (329.4 mg, 91%). To the suspension of CuI (19.0 mg, 0.1 mmol), L-proline (23.0 mg, 0.2 mmol), and K₂CO₃ (345.5 mg, 2.5 mmol) in 2-methylethanol (5 mL) in a microwave tube was added 2-((2-bromo-4-methylphenyl)thio)-5-(trifluoromethyl)aniline (181.1 mg, 0.5 mmol). After being stirred for 2 h at 160° C. under microwave irradiation, the mixture was concentrated and purified by silica gel flash chromatography (10-50% ethyl acetate in hexane) to give 2-methyl-8-(trifluoromethyl)-10H-phenothiazine as oil.

To the suspension of 2-methyl-8-(trifluoromethyl)-10H-phenothiazine (56.2 mg, 0.2 mmol) and sodium hydride (24 mg, 60% in mineral oil, 0.6 mmol) in DMF (2 mL) was added 2-(1-methylpiperidin-2-yl)ethyl 4-methylbenzenesulfonate (119 mg, 0.4 mmol). After being stirred for 1 h at 50° C., water was added, and the mixture was extracted with ethyl acetate (5 mL×3), dried over Na₂SO₄, filtered, and concentrated. The resulting mixture was purified by preparative HPLC (10-100% methanol/0.1% TFA in H₂O) to give the title compound as oil in its TFA salt form (42.1 mg, 40%). ¹H NMR (600 MHz, Methanol-d₄) δ 7.29 (d, J=8.0 Hz, 1H), 7.23-7.18 (m, 2H), 7.03 (dd, J=7.8, 1.6 Hz, 1H), 6.89 (d, J=4.1 Hz, 1H), 6.83 (d, J=7.8 Hz, 1H), 4.13 (dt, J=12.1, 6.0 Hz, 1H), 4.05 (ddd, J=14.2, 8.1, 6.0 Hz, 1H), 3.49 (d, J=11.6 Hz, 0.3H), 3.44-3.38 (m, 0.7H), 3.17-3.01 (m, 1.3H), 2.96-2.89 (m, 0.7H), 2.67 (s, 2H), 2.62 (s, 1H), 2.49-2.42 (m, 0.7H), 2.29 (s, 3H), 2.17-2.10 (m, 0.3H), 2.07-1.98 (m, 1H), 1.89 (ddt, J=14.8, 9.4, 5.7 Hz, 1H), 1.86-1.73 (m, 2H), 1.66 (dtt, J=16.3, 13.5, 3.8 Hz, 1.3H), 1.56-1.38 (m, 1.7H). HRMS m/z [M+H]⁺ calcd for C₂₂H₂₆F₃N₂S⁺ 407.1763, found 407.1771.

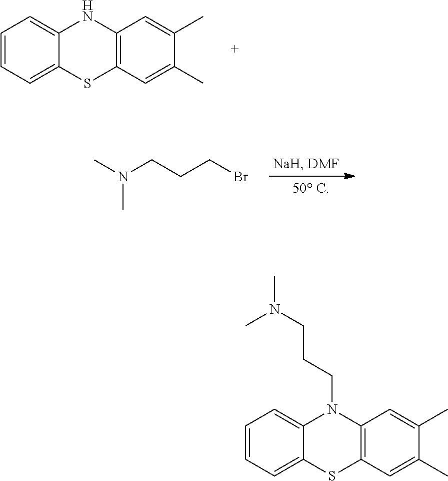

Example 12: 3-(2,3-dimethyl-10H-phenothiazin-10-yl)-N,N-dimethylpropan-1-amine (TC₈₄-124): To the suspension of 2,3-dimethyl-10H-phenothiazine (22.7 mg, 0.1 mmol) and sodium hydride (24 mg, 60% in mineral oil, 0.6 mmol) in DMF (2 mL) was added 3-bromo-N,N-dimethylpropan-1-amine hydrobromide (49.4 mg, 0.2 mmol). After being stirred for 1 h at 50° C., water was added, and the mixture was extracted with ethyl acetate (5 mL×3), dried over Na₂SO₄, filtered, and concentrated. The resulting mixture was purified by preparative HPLC (10-100% methanol/0.1% TFA in H₂O) to give the title compound as oil in its TFA salt form (15.9 mg, 37%). ¹H NMR (600 MHz, Methanol-d₄) δ 7.24-7.20 (m, 1H), 7.17 (dd, J=7.6, 1.5 Hz, 1H), 7.06 -7.00 (m, 1H), 6.99-6.93 (m, 2H), 6.84 (s, 1H), 4.07 (s, 2H), 3.26-3.20 (m, 2H), 2.79 (s, 6H), 2.25 (s, 3H), 2.12-2.15 (m, 5H). HRMS m/z [M+H]⁺ calcd for C₁₉H₂₅N₂S⁺ 313.1733, found 313.1738.

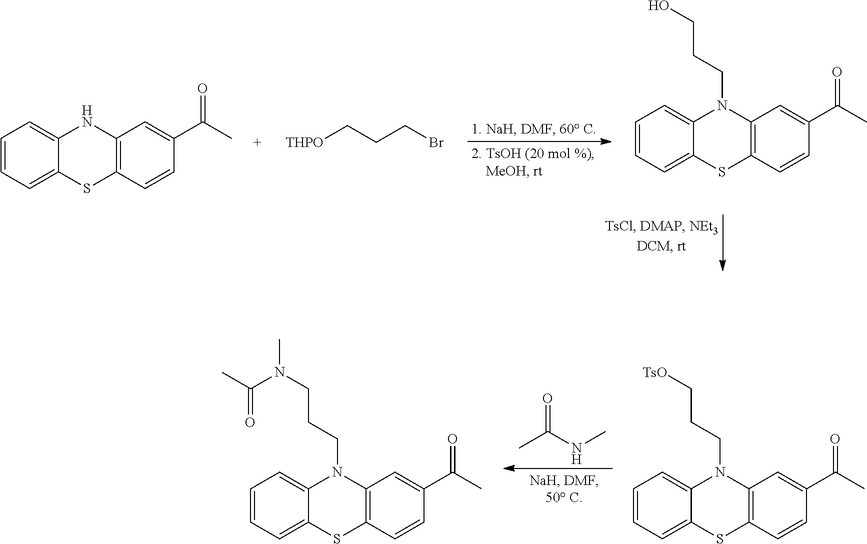

Example 13: N-(3-(2-acetyl-10H-phenothiazin-10-yl)propyl)-N-methylacetamide (TC₈₄-161): To the suspension of 1-(10H-phenothiazin-2-yl)ethan-1-one (241.3 mg, 1 mmol) and sodium hydride (60 mg, 60% in mineral oil, 1.5 mmol) in DMF (4 mL) was added 2-(3-bromopropoxy)tetrahydro-2H-pyran (334.7 mg, 1.5 mmol). After being stirred for 0.5 h at 60° C., water was added, and the mixture was extracted with ethyl acetate (5 mL×3), dried over Na₂SO₄, filtered, and concentrated. To the resulting crude mixture was added TsOH·H₂O (38 mg, 0.2 mmol) and MeOH (4 mL). After being stirred for 2 h at room temperature, the resulting mixture was purified by silica gel flash chromatography (0-100% ethyl acetate in hexane) to give 1-(10-(3-hydroxypropyl)-10H-phenothiazin-2-yl)ethan-1-one as oil (209.1 mg, 70%).

To the solution of 1-(10-(3-hydroxypropyl)-10H-phenothiazin-2-yl)ethan-1-one (209.1 mg, 0.7 mmol), DMAP (24.4 mg, 0.2 mmol), and tosyl chloride (286.0 mg, 1.5 mmol) in DCM (5 mL) was added NEt3 (0.3 mL, 2 mmol). After being stirred for 2 h, the resulting mixture was concentrated and purified by silica gel flash chromatography (0-100% ethyl acetate in hexane) to give 3-(2-acetyl-10H-phenothiazin-10-yl)propyl 4-methylbenzenesulfonate as oil. To the suspension of 3-(2-acetyl- 1 OH-phenothiazin-10-yl)propyl 4-methylbenzenesulfonate and sodium hydride (60 mg, 60% in mineral oil, 1.5 mmol) in DMF (4 mL) was added N-methylacetamide (146.2 mg, 2 mmol). After being stirred for 0.5 h at 50° C., water was added, and the mixture was extracted with ethyl acetate (5 mL×3), dried over Na₂SO₄, filtered, and concentrated. The crude product was purified by reverse-phase ISCO (10-100% methanol/0.1% TFA in H₂O) to give the title compound as oil (88.9 mg, 25%). ¹H NMR (600 MHz, Methanol-d₄) δ 7.66-7.56 (m, 1H), 7.54 (d, J=1.7 Hz, 1H), 7.31-7.18 (m, 2H), 7.16-6.94 (m, 3H), 4.07 (dt, J=17.0, 6.4 Hz, 2H), 4.03-3.98 (m, 1H), 3.72 (t, J=6.1 Hz, 1H), 3.50 (dt, J=15.1, 7.2 Hz, 1H), 3.37 (s, 2H), 2.99 (s, 1.5H), 2.84 (d, J=2.9 Hz, 1.5H), 2.62-2.58 (m, 3H), 2.13-2.05 (m, 1H), 1.85 (d, J=3.4 Hz, 1H). HRMS m/z [M+H]⁺ calcd for C₂₀H₂₃N₂O₂S⁺ 355.1475, found 355.1477.

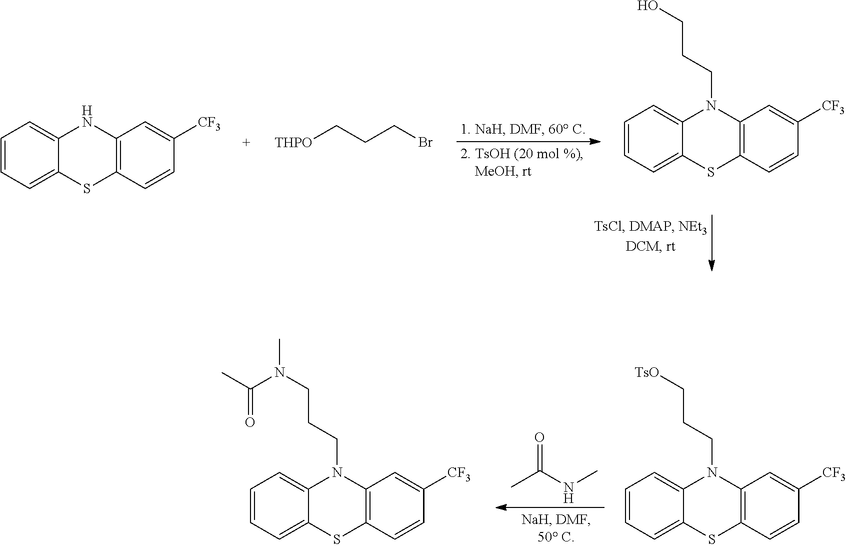

Example 14: N-methyl-N-(3-(2-(trifluoromethyl)-10H-phenothiazin-10-yl)propyl)acetamide (TC₈₄-162): To the suspension of 2-(trifluoromethyl)-10H-phenothiazine (267.3 mg, 1 mmol) and sodium hydride (60 mg, 60% in mineral oil, 1.5 mmol) in DMF (4 mL) was added 2-(3-bromopropoxy)tetrahydro-2H-pyran (334.7 mg, 1.5 mmol). After being stirred for 0.5 h at 60° C., water was added, and the mixture was extracted with ethyl acetate (5 mL×3), dried over Na₂SO₄, filtered, and concentrated. To the resulting crude mixture was added TsOH·H₂O (38 mg, 0.2 mmol) and MeOH (4 mL). After being stirred for 2 h at room temperature, the resulting mixture was purified by silica gel flash chromatography (0-100% ethyl acetate in hexane) to give 3-(2-(trifluoromethyl)-10H-phenothiazin-10-yl)propan-1-ol as oil (324.5 mg, 100%). To the solution of 3-(2-(trifluoromethyl)-10H-phenothiazin-10-yl)propan-1-ol (324.5 mg, 1 mmol), DMAP (24.4 mg, 0.2 mmol), and tosyl chloride (286.0 mg, 1.5 mmol) in DCM (5 mL) was added NEt₃ (0.3 mL, 2 mmol). After being stirred for 2 h, the resulting mixture was concentrated and purified by silica gel flash chromatography (0-100% ethyl acetate in hexane) to give 3 -(2-(trifluoro methyl)-10H-phenothiazin-10-yl)propyl 4-methylbenzenesulfonate as oil. To the suspension of 3 -(2-(trifluoromethyl)-10H-phenothiazin-10-yl)propyl 4-methylbenzenesulfonate and sodium hydride (60 mg, 60% in mineral oil, 1.5 mmol) in DMF (4 mL) was added N-methylacetamide (146.2 mg, 2 mmol). After being stirred for 0.5 h at 50° C., water was added, and the mixture was extracted with ethyl acetate (5 mL×3), dried over Na₂SO₄, filtered, and concentrated. The crude product was purified by reverse-phase ISCO (10-100% methanol/0.1% TFA in H₂O) to give the title compound as oil (192.8 mg, 51%). ¹H NMR (600 MHz, Methanol-d₄) δ 7.34 (d, J=8.0 Hz, 1H), 7.32-7.14 (m, 4H), 7.11-6.98 (m, 2H), 4.04 (t, J=6.0 Hz, 1H), 3.99 (t, J=6.6 Hz, 1H), 3.52-3.44 (m, 2H), 2.95 (s, 1.5H), 2.82 (s, 1.5H), 2.11-1.99 (m, 3.5H), 1.83 (s, 1.5H). HRMS m/z [M+H]⁺ calcd for C₁₉H₂₀F₃N₂OS⁺ 381.1243, found 381.1238.

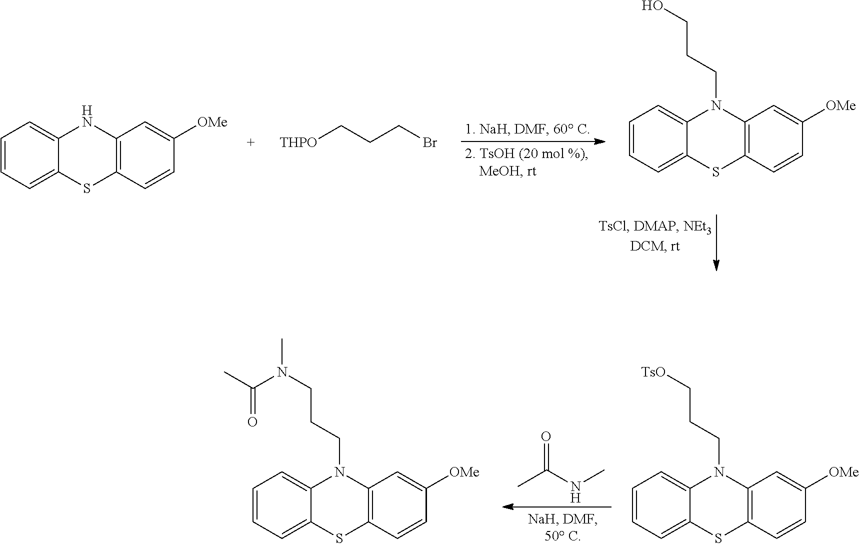

Example 15: N-(3-(2-methoxy-10H-phenothiazin-10-yl)propyl)-N-methylacetamide (TC₈₄-163): To the suspension of 2-methoxy-10H-phenothiazine (229.3 mg, 1 mmol) and sodium hydride (60 mg, 60% in mineral oil, 1.5 mmol) in DMF (4 mL) was added 2-(3-bromopropoxy)tetrahydro-2H-pyran (334.7 mg, 1.5 mmol). After being stirred for 0.5 h at 60° C., water was added, and the mixture was extracted with ethyl acetate (5 mL×3), dried over Na₂SO₄, filtered, and concentrated. To the resulting crude mixture was added TsOH·H₂O (38 mg, 0.2 mmol) and MeOH (4 mL). After being stirred for 2 h at room temperature, the resulting mixture was purified by silica gel flash chromatography (0-100% ethyl acetate in hexane) to give 3-(2-methoxy-10H-phenothiazin-10-yl)propan-1-ol as oil (246.0 mg, 80%).

To the solution of 3-(2-methoxy-10H-phenothiazin-10-yl)propan-1-ol (246.0 mg, 0.8 mmol), DMAP (24.4 mg, 0.2 mmol), and tosyl chloride (286.0 mg, 1.5 mmol) in DCM (5 mL) was added NEt 3 (0.3 mL, 2 mmol). After being stirred for 2 h, the resulting mixture was concentrated and purified by silica gel flash chromatography (0-100% ethyl acetate in hexane) to give 3-(2-methoxy-10H-phenothiazin-10-yl)propyl 4-methylbenzenesulfonate as oil. To the suspension of 3-(2-methoxy-10H-phenothiazin-10-yl)propyl 4-methylbenzenesulfonate and sodium hydride (60 mg, 60% in mineral oil, 1.5 mmol) in DMF (4 mL) was added N-methylacetamide (146.2 mg, 2 mmol). After being stirred for 0.5 h at 50° C., water was added, and the mixture was extracted with ethyl acetate (5 mL×3), dried over Na₂SO₄, filtered, and concentrated. The crude product was purified by reverse-phase ISCO (10-100% methanol/0.1% TFA in H₂O) to give the title compound as oil (176.7 mg, 52%). i H NMR (600 MHz, Methanol-d 4) δ 7.22-7.10 (m, 2H), 7.06-6.89 (m, 3H), 6.60-6.50 (m, 2H), 3.94-3.85 (m, 2H), 3.77 (s, 3H), 3.44 (t, J=6.9 Hz, 1H), 3.39 (t, J=7.4 Hz, 1H), 2.89 (s, 1,5H), 2.79 (s, 1.5H), 2.04-1.96 (m, 3.5H), 1.79 (s, 1.5H). HRMS m/z [M+H]⁺ calcd for C₁₉H₂₃N₂O₂S⁺ 343.1475, found 343.1470.

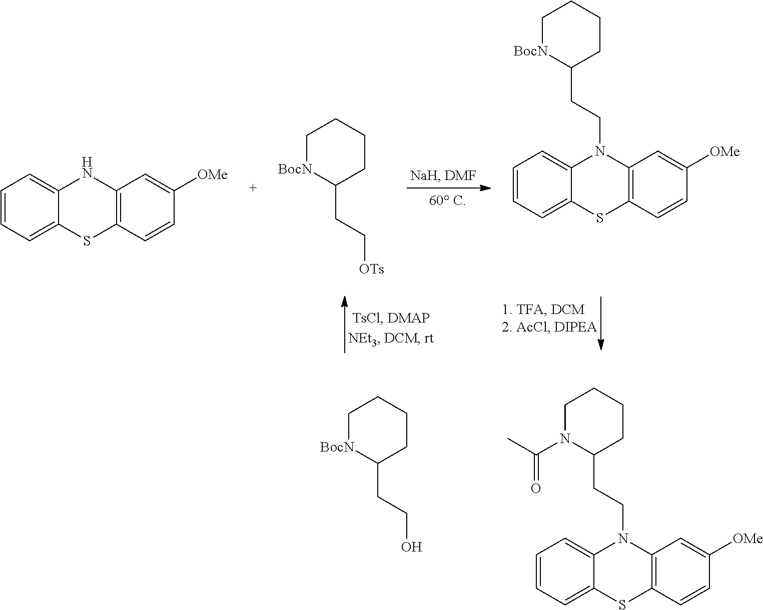 [SMILES: COc1ccc2c(c1)Nc1ccccc1S2 ; Cc1ccc(S(=O)(=O)OCCC2CCCCN2C(=O)OC(C)(C)C)cc1 ; COc1ccc2c(c1)N(CCC1CCCCN1C(=O)OC(C)(C)C)c1ccccc1S2 ; CC(C)(C)OC(=O)N1CCCCC1CCO ; COc1ccc2c(c1)N(CCC1CCCCN1C(C)=O)c1ccccc1S2]

Example 16:1-(2-(2-(2-methoxy-10H-phenothiazin-10-yl)ethyl)piperidin-1-yl)ethan-1-one (TC₉₆-32): tent-butyl 2-(2-(tosyloxy)ethyl)piperidine-1-carboxylate: To the solution of tent-butyl 2-(2-hydroxyethyl)piperidine-1-carboxylate (458.6 mg, 2 mmol), DMAP (48.8 mg, 0.4 mmol), and tosyl chloride (571.8 mg, 3 mmol) in DCM (5 mL) was added NEt₃ (0.55 mL, 4 mmol). After being stirred for 2 h, the resulting mixture was concentrated and purified by silica gel flash chromatography (0-100% ethyl acetate in hexane) to give the title compound as oil (407.5 mg, 53%).

To the suspension of 2-methoxy-10H-phenothiazine (229.3 mg, 1 mmol) and sodium hydride (60 mg, 60% in mineral oil, 1.5 mmol) in DMF (4 mL) was added tent-butyl 2-(2-(tosyloxy)ethyl)piperidine- 1 -carboxylate (407.5 mg, 1.06 mmol). After being stirred for 1 h at 60° C., water was added, and the mixture was extracted with ethyl acetate (10 mL×3), dried over Na₂SO₄, filtered, and concentrated. The resulting mixture was purified by silica gel flash chromatography (0-100% ethyl acetate in hexane) to give tent-butyl 2-(2-(2-methoxy-10H-phenothiazin-10-yl)ethyl)piperidine-1-carboxylate as oil. To the solution of tent-butyl 2-(2-(2-methoxy-10H-phenothiazin-10-yl)ethyl)piperidine- 1 -carboxylate in DCM (5 mL) was added TFA (2 mL). After being stirred for 2 h, the resulting mixture was concentrated. To the crude mixture were added DCM (5 mL), AcC₁ (0.14 mL, 2 mmol), and DIPEA (1.74 mL, 10 mmol). After being stirred for 1 h at room temperature, the resulting mixture was concentrated and purified by reverse-phase ISCO (10-100% methanol/0.1% TFA in H₂O) to give the title compound as oil (239.3 mg, 63%). ¹H NMR (600 MHz, Methanol-d 4) δ 7.23-7.10 (m, 2H), 7.07-6.99 (m, 2H), 6.99-6.90 (m, 1H), 6.61-6.56 (m, 1H), 6.52-6.48 (m, 1H), 4.40 (dd, J=13.5, 4.5 Hz, 1H), 4.19 (p, J=5.5 Hz, 1H), 3.91 (s, 1.5H), 3.79 (s, 3H), 3.67 (d, J=13.5 Hz, 0.5H), 3.14 (td, J=13.5, 2.9 Hz, 0.5H), 2.66 (td, J=13.4, 2.8 Hz, 0.5H), 2.24-2.07 (m, 1.5H), 1.96 (s, 1.5H), 1.73 (s, 2H), 1.70-1.51 (m, 4.5H), 1.31 (d, J=8.5 Hz, 0.5H). HRMS m/z [M+H]⁺ calcd for C₂₂H₂₇N₂O₂S⁺ 383.1788, found 383.1790.

Certain compounds disclosed herein have the structures shown in Table 1.

TABLE 1

| Example | Compound | | |
|---|---|---|---|
| # | ID | Structure | Name |
| 1 | TC84-31 | 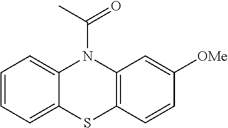 [SMILES: COc1ccc2c(c1)N(C(C)=O)c1ccccc1S2] | 1-(2-methoxy-10H-phenothiazin-10- yl)ethan-1-one |
| 2 | TC84-37 | 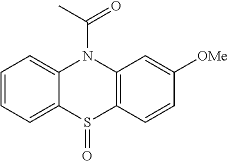 [SMILES: COc1ccc2c(c1)N(C(C)=O)c1ccccc1S2=O] | 1-(2-methoxy-5-oxido-10H- phenothiazin-10-yl)ethan-1-one |
| 3 | TC84-32 | 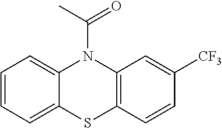 [SMILES: CC(=O)N1c2ccccc2Sc2ccc(C(F)(F)F)cc21] | 1-(2-(trifluoromethyl)-10H- phenothiazin-10-yl)ethan-1-one |
| 4 | TC84-38 | 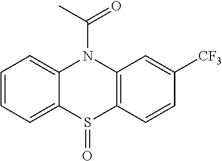 [SMILES: CC(=O)N1c2ccccc2S(=O)c2ccc(C(F)(F)F)cc21] | 1-(5-oxido-2-(trifluoromethyl)-10H- phenothiazin-10-yl)ethan-1-one |
| 5 | TC84-13 | 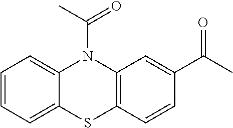 [SMILES: CC(=O)c1ccc2c(c1)N(C(C)=O)c1ccccc1S2] | 1,1′-(10H-phenothiazine-2,10- diyl)bis(ethan-1-one) |
| 6 | TC84-20 | 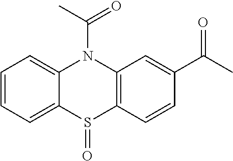 [SMILES: CC(=O)c1ccc2c(c1)N(C(C)=O)c1ccccc1S2=O] | 1,1′-(5-oxido-10H-phenothiazine- 2,10-diyl)bis(ethan-1-one) |
| 7 | TC84-39 | 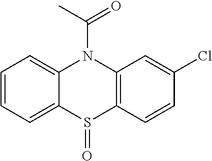 [SMILES: CC(=O)N1c2ccccc2S(=O)c2ccc(Cl)cc21] | 1-(2-chloro-5-oxido-10H- phenothiazin-10-yl)ethan-1-one |
| 8 | TC84-110 | 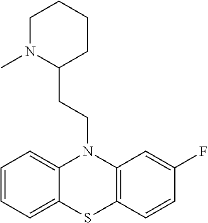 [SMILES: CN1CCCCC1CCN1c2ccccc2Sc2ccc(F)cc21] | 2-fluoro-10-(2-(1-methylpiperidin-2- yl)ethyl)-10H-phenothiazine |
| 9 | TC84-144 | 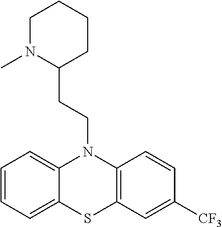 [SMILES: CN1CCCCC1CCN1c2ccccc2Sc2cc(C(F)(F)F)ccc21] | 10-(2-(1-methylpiperidin-2-yl)ethyl)- 3-(trifluoromethyl)-10H- phenothiazine |
| 10 | TC84-125 | 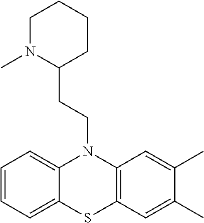 [SMILES: Cc1cc2c(cc1C)N(CCC1CCCCN1C)c1ccccc1S2] | 2,3-dimethyl-10-(2-(1- methylpiperidin-2-yl)ethyl)-10H- phenothiazine |
| 11 | TC96-20 | 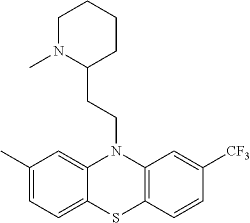 [SMILES: Cc1ccc2c(c1)N(CCC1CCCCN1C)c1cc(C(F)(F)F)ccc1S2] | 2-methyl-10-(2-(1-methylpiperidin-2- yl)ethyl)-8-(trifluoromethyl)-10H- phenothiazine |
| 12 | TC84-124 | 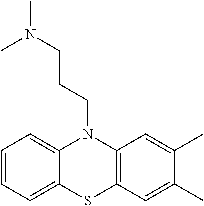 [SMILES: Cc1cc2c(cc1C)N(CCCN(C)C)c1ccccc1S2] | 3-(2,3-dimethyl-10H-phenothiazin- 10-yl)-N,N-dimethylpropan-1-amine |
| 13 | TC84-161 | 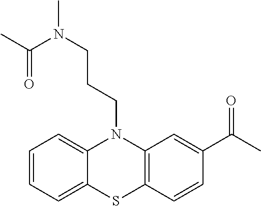 [SMILES: CC(=O)c1ccc2c(c1)N(CCCN(C)C(C)=O)c1ccccc1S2] | N-(3-(2-acetyl-10H-phenothiazin-10- yl)propyl)-N-methylacetamide |
| 14 | TC84-162 | 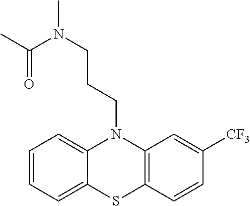 [SMILES: CC(=O)N(C)CCCN1c2ccccc2Sc2ccc(C(F)(F)F)cc21] | N-methyl-N-(3-(2-(trifluoromethyl)- 10H-phenothiazin-10- yl)propyl)acetamide |
| 15 | TC84-163 | 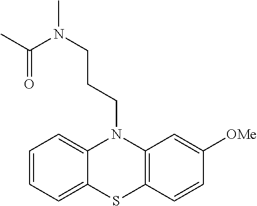 [SMILES: COc1ccc2c(c1)N(CCCN(C)C(C)=O)c1ccccc1S2] | N-(3-(2-methoxy-10H-phenothiazin- 10-yl)propyl)-N-methylacetamide |
| 16 | TC96-32 | 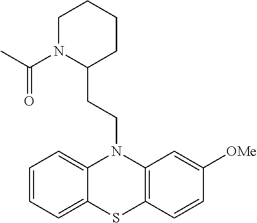 [SMILES: COc1ccc2c(c1)N(CCC1CCCCN1C(C)=O)c1ccccc1S2] | 1-(2-(2-(2-methoxy-10H- phenothiazin-10-yl)ethyl)piperidin-1- yl)ethan-1-one |

Compounds corresponding to Examples 1-16 have been synthesized and are provided with a Compound Code in Table 1.

As used herein, in case of discrepancy between the structure and chemical name provided for a particular compound, the given structure shall control.

### Example 17

### Structures and Numerical Values of Novel Compositions of Matter Screened for Protective Properties Relevant to Alzheimer's and Other Age-Related Diseases. (Table 2)

Compounds TC₈₄-110; TC₈₄-125; TC₈₄-124; TC₈₄-162; TC₉₆-20 significantly reduced both microglial TNF-alpha secretion and reduced Abeta toxicity (Table 2). Several of these compounds (#TC₈₄-125, TC₈₄-144, TC₈₄-124, TC₈₄-110, TC₈₄-161, TC₈₄-39, TC₈₄-13, TC₈₄-20, TC₈₄-38, and TC₈₄-163) also increased lifespan in C. elegans, indicating general protective effects during aging.

TABLE 2
Structures and numerical values of novel compositions of matter screened for
protective properties relevant to Alzeheimer’s and other age-related diseases. The following
table depicts the screened compound Identification number (ID#), the percentage of TNFa vs LPS
stimulated controls (TNFa in blue), the corresponding standard error of the mean for TNFa (TNFa
SEM), the percentage of MTT viability compared to unstimulated DMSO control cells (MTT in
red), the corresponding standard error of the mean for MTT (MTT SEM) and the structure for the
compound being screened (Structure).

| Example | Compound | | | | | TNFa | MTT | |
|---|---|---|---|---|---|---|---|---|
| # | ID | TNFa | MTT | Paralysis | Lifespan | SEM | SEM | Structure |
| 10 | TC84-125 | 41.37% | 42.77% | 64.89% 75.6% | I | 4.81% | 10.58% | 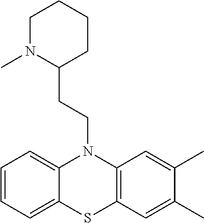 [SMILES: Cc1cc2c(cc1C)N(CCC1CCCCN1C)c1ccccc1S2] |
| 9 | TC84-144 | 45.34% | 41.27% | 25.88% 84.3% | I | 4.64% | 9.05% | 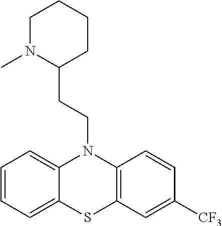 [SMILES: CN1CCCCC1CCN1c2ccccc2Sc2cc(C(F)(F)F)ccc21] |
| 11 | TC96-20 | 49.67% | 51.90% | 69.24% 80.5% | | 6.78% | 12.06% | 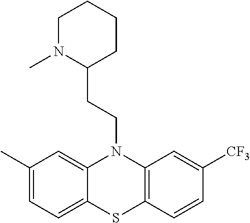 [SMILES: Cc1ccc2c(c1)N(CCC1CCCCN1C)c1cc(C(F)(F)F)ccc1S2] |
| 12 | TC84-124 | 56.73% | 82.48% | 66.50% 54.3% | I | 3.69% | 8.22% | 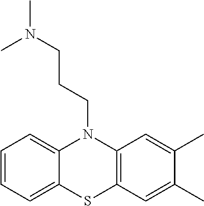 [SMILES: Cc1cc2c(cc1C)N(CCCN(C)C)c1ccccc1S2] |
| | DMSO | 57.24% | 100.00% | 100% | | 1.90% | 3.15% | DMSO |
| 8 | TC84-110 | 58.49% | 91.99% | 70.72% 48.6% | I | 5.23% | 5.49% | 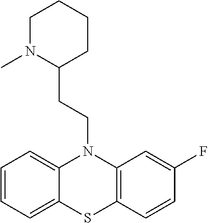 [SMILES: CN1CCCCC1CCN1c2ccccc2Sc2ccc(F)cc21] |
| 16 | TC96-32 | 78.98% | 106.92% | 99.47% 82.97% | | 1.79% | 5.62% | 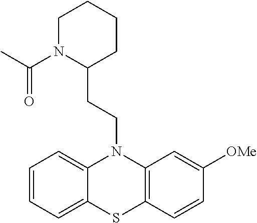 [SMILES: COc1ccc2c(c1)N(CCC1CCCCN1C(C)=O)c1ccccc1S2] |
| 13 | TC84-161 | 89.27% | 106.15% | 86.69% 107.66% | I | 2.17% | 3.94% | 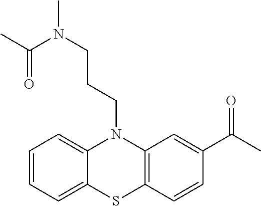 [SMILES: CC(=O)c1ccc2c(c1)N(CCCN(C)C(C)=O)c1ccccc1S2] |
| 2 | TC84-37 | 90.49% | 140.38% | 140.76% 102.83% | D | 2.51% | 10.49% | 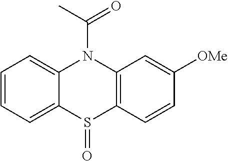 [SMILES: COc1ccc2c(c1)N(C(C)=O)c1ccccc1S2=O] |
| 3 | TC84-32 | 90.74% | 141.59% | 138.30% 105.34 | N | 1.97% | 16.79% | 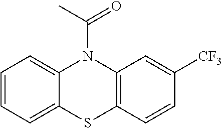 [SMILES: CC(=O)N1c2ccccc2Sc2ccc(C(F)(F)F)cc21] |
| 7 | TC84-39 | 90.88% | 134.67% | 139.39% 84.27% | I | 3.98% | 11.59% | 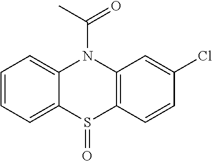 [SMILES: CC(=O)N1c2ccccc2S(=O)c2ccc(Cl)cc21] |
| 1 | TC84-31 | 92.28% | 153.72% | 141.28% 52.16% | N | 2.65% | 21.15% | 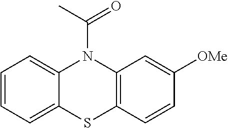 [SMILES: COc1ccc2c(c1)N(C(C)=O)c1ccccc1S2] |
| 14 | TC84-162 | 94.58% | 121.08% | 69.06% 65.68% | N | 1.53% | 9.01% | 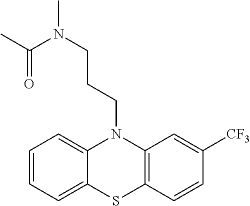 [SMILES: CC(=O)N(C)CCCN1c2ccccc2Sc2ccc(C(F)(F)F)cc21] |
| 5 | TC84-13 | 95.36% | 154.03% | 143.91% 107.5% | I | 3.19% | 22.07% | 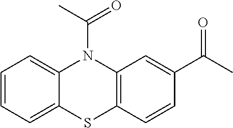 [SMILES: CC(=O)c1ccc2c(c1)N(C(C)=O)c1ccccc1S2] |
| 6 | TC84-20 | 95.41% | 158.76% | 109.0% 95.66% | I | 3.05% | 23.69% | 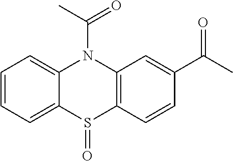 [SMILES: CC(=O)c1ccc2c(c1)N(C(C)=O)c1ccccc1S2=O] |
| 4 | TC84-38 | 97.23% | 149.90% | 123.81% 60.86% | I | 2.51% | 15.24% | 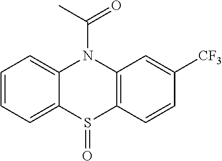 [SMILES: CC(=O)N1c2ccccc2S(=O)c2ccc(C(F)(F)F)cc21] |
| 15 | TC84-163 | 99.98% | 108.79% | 96.43% 67.3% | I | 3.41% | 4.97% | 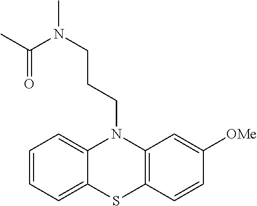 [SMILES: COc1ccc2c(c1)N(CCCN(C)C(C)=O)c1ccccc1S2] |
| | LPS | 100.00% | 96.84% | | | 1.25% | 5.24% | LPS |

### Example 18

### Effects of Novel Compounds and Controls to Inhibit SARS-CoV-2 Replication (Table 3)

Because these compounds show such promising effects to reduce age-related impairments, in particular inflammatory secretion of TNF-alpha, a major driver of cytokine storms that mediate

COVID-19 mortality, and because COVID-19 produces mortality exponentially dependent on age, efficacy of several of the novel compounds (and some controls and known phenothiazines) were assessed for efficacy to reduce SARS-CoV-2 replication. Remarkably, all of the novel compounds tested (TC₈₄-110; TC₈₄-124) also reduced SARS-CoV-2 replication, some as effectively as Remdesivir, the current gold standard for treating COVID-19 based on inhibition of coronavirus replication.

TABLE 3
Effects of novel compounds and controls to inhibit SARS-CoV-2 replication. This
table, using the same numbering codes as in Table 1, indicated the concentration at which each
drug inhibits the replication of SARS-CoV-2 in Vero cells by 50% (IC50) and the concentration
at which each drugs reduces viability of Vero cells (CC50). These studies were carried out under
the direction of Kris White and Adolfo Garcia-Sastre in the Microbiology Department using coded
samples. TNF-a (microglial inflammation), MTT (microglial toxicity), paralysis in C. elegans
(Abeta toxicity) and Lifespan in C. elegans (net protective effects) are presented for comparison.

| | | | | | | IC₅₀ | CC₅₀ | |
|---|---|---|---|---|---|---|---|---|
| # | ID/Name | TNF-a. | MTT | paralysis | Lifespan | (uM) | (uM) | Structure |
| NA | Remdesivir | NA | NA | NA | NA | 1.2 | >20 | |
| NA | Chlorpromazine | 63.83% | 79.38% | 0% | I | 4.4 | 6.7 | 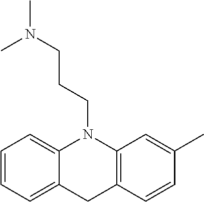 [SMILES: Cc1ccc2c(c1)N(CCCN(C)C)c1ccccc1C2] |
| 75 | Triflupromazine Hydrochloride | 62.95% | 98.95% | 23.58% 67.08% 62.19% 8.708% 10.073% 51.3% | I <0.0001 | 2.5 | 7.0 | 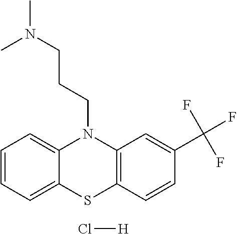 |
| 45 | Chlorprothixene | 69.84% | 122.40% | 64.31% 47.65% | I 0.0493 | 1 | 3.5 | 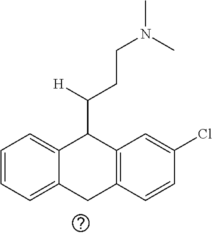 [SMILES: CN(C)CCCC1c2ccccc2Cc2ccc(Cl)cc21] |
| 8 | TC84-110 | 58.49% | 91.99% | 70.72% 28.84% | I | 1.2 | 8.1 | 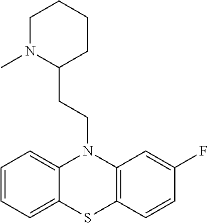 [SMILES: CN1CCCCC1CCN1c2ccccc2Sc2ccc(F)cc21] |
| 12 | TC84-124 | 56.73% | 82.48% | 66.50% 32.22% | I | 1.7 | 6.5 | 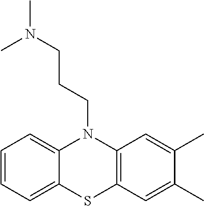 [SMILES: Cc1cc2c(cc1C)N(CCCN(C)C)c1ccccc1S2] |

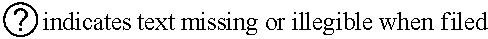

### Example 19

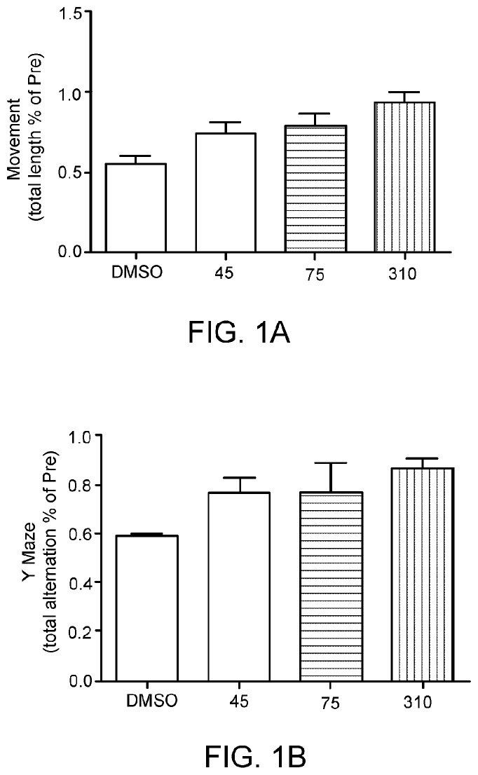

### Phenothiazines Preserve Motor and Cognitive Function in a Mouse Model of Intracerebral Hemorrhage (FIG. 1)

TC84-110 (#310)was assessed for protection in a mouse model of intracerebral hemorrhage and was significantly protective to maintain open field behavior and cognitive impairments when administered after the lesion was produced (FIG. 1). This compound also produced no discernable off-target effects such as sedation, a common off-target effect of pharmacologically active phenothiazines. Compounds #45 (Chlorprothixen) #75 (Triflupromazine) and #310 (example 8/TC₈₄-110) preserve motor and cognitive function compared to DMSO. Motor function was assessed using rotarod and and cognitive was assessed by alternation in a Y-maze. All data were normalized to data pre-ICH function.

### Materials and Methods

### General Chemistry Methods

For the synthesis of intermediates and examples (1-89) below, HPLC spectra for all compounds were acquired using an Agilent 1200 Series system with DAD detector. Chromatography was performed on a 2.1×150 mm Zorbax 300SB-C18 5 μm column with water containing 0.1% formic acid as solvent A and acetonitrile containing 0.1% formic acid as solvent B at a flow rate of 0.4 ml/min. The gradient program was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Nuclear Magnetic Resonance (NMR) spectra were acquired on a Bruker DRX-600 spectrometer with 600 MHz for proton (¹H NMR) and 150 MHz for carbon (¹³C NMR); chemical shifts are reported in (δ). Preparative HPLC was performed on Agilent Prep 1200 series with UV detector set to 254 nm. Samples were injected onto a Phenomenex Luna 250×30 mm, 5 μm, C₁₈ column at room temperature. The flow rate was 40 ml/min. A linear gradient was used with 10% (or 50%) of MeOH (A) in H₂O (with 0.1% TFA) (B) to 100% of MeOH (A). HPLC was used to establish the purity of target compounds. All final compounds had >95% purity using the HPLC methods described above.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Alzheimer's, A. (2016). “2016 Alzheimer's disease facts and figures.” Alzheimers Dement 12(4): 459-509.

Cheong, C. U., C. P. Chang, C. M. Chao, B. C. Cheng, C. Z. Yang and C. C. Chio (2013). “Etanercept attenuates traumatic brain injury in rats by reducing brain TNF- alpha contents and by stimulating newly formed neurogenesis.” Mediators Inflamm 2013: 620837.

Chio, C. C., C. H. Chang, C. C. Wang, C. U. Cheong, C. M. Chao, B. C. Cheng, C. Z. Yang and C. P. Chang (2013). “Etanercept attenuates traumatic brain injury in rats by reducing early microglial expression of tumor necrosis factor-alpha.” BMC Neurosci 14: 33.

de Pedro-Cuesta, J., A. Rabano, P. Martinez-Martin, M. Ruiz-Tovar, E. Alcalde-Cabero, J. Almazan-Isla, F. Avellanal and M. Calero (2015). “Comparative Incidence of Conformational, Neurodegenerative Disorders.” PLoS One 10(9): e0137342.

Decourt, B., D. K. Lahiri and M. N. Sabbagh (2017). “Targeting Tumor Necrosis Factor Alpha for Alzheimer's Disease.” Curr Alzheimer Res 14(4): 412-425.

Del Valle, D. M., S. Kim-Schulze, H. H. Huang, N. D. Beckmann, S. Nirenberg, B. Wang, Y. Lavin, T. H. Swartz, D. Madduri, A. Stock, T. U. Marron, H. Xie, M. Patel, K. Tuballes, O. Van Oekelen, A. Rahman, P. Kovatch, J. A. Aberg, E. Schadt, S. Jagannath, M. Mazumdar, A. W. Charney, A. Firpo-Betancourt, D. R. Mendu, J. Jhang, D. Reich, K. Sigel, C. Cordon-Cardo, M. Feldmann, S. Parekh, M. Merad and S. Gnjatic (2020). “An inflammatory cytokine signature predicts COVID-19 severity and survival.” Nat Med.

Dobbs, R. J., A. Charlett, A. G. Purkiss, S. M. Dobbs, C. Weller and D. W. Peterson (1999). “Association of circulating TNF-alpha and IL-6 with ageing and parkinsonism.” Acta Neurol Scand 100(1): 34-41.

Eleti, S. (2016). “Drugs in Alzheimer's disease Dementia: An overview of current pharmacological management and future directions.” Psychiatr Danub 28(Supp1-1): 136-140.

Evason, K., C. Huang, I. Yamben, D. F. Covey and K. Kornfeld (2005). “Anticonvulsant medications extend worm life-span.” Science 307(5707): 258-262.

Franceschi, C., M. Bonafe, S. Valensin, F. Olivieri, M. De Luca, E. Ottaviani and G. De Benedictis (2000). “Inflamm-aging. An evolutionary perspective on immunosenescence.” Ann N Y Acad Sci 908: 244-254.

Govindaraj an, N., R. C. Agis-Balboa, J. Walter, F. Sananbenesi and A. Fischer (2011). “Sodium butyrate improves memory function in an Alzheimer's disease mouse model when administered at an advanced stage of disease progression.” J Alzheimers Dis 26(1): 187-197.

Greig, S. L. (2015). “Memantine ER/Donepezil: A Review in Alzheimer's Disease.” CNS Drugs 29(11): 963-970.

Hodgson, J. G., N. Agopyan, C. A. Gutekunst, B. R. Leavitt, F. LePiane, R. Singaraja, D. J. Smith, N. Bissada, K. McCutcheon, J. Nasir, L. Jamot, X. J. Li, M. E. Stevens, E. Rosemond, J. C. Roder, A. G. Phillips, E. M. Rubin, S. M. Hersch and M. R. Hayden (1999). “A YAC mouse model for Huntington's disease with full-length mutant huntingtin, cytoplasmic toxicity, and selective striatal neuro degeneration.” Neuron 23(1): 181-192.

Holmes, C., C. Cunningham, E. Zotova, D. Culliford and V. H. Perry (2011). “Proinflammatory cytokines, sickness behavior, and Alzheimer disease.” Neurology 77(3): 212-218.

Kaur, D., V. Sharma and R. Deshmukh (2019). “Activation of microglia and astrocytes: a roadway to neuroinflammation and Alzheimer's disease.” Inflammopharmacology 27(4): 663-677.

Ladika, S. (2019). “Bringing Humira (Its Price) Down a Peg.” Manag Care 28(1): 7-8.

Link, C. D. (2001). “Transgenic invertebrate models of age-associated neurodegenerative diseases.” Mech Ageing Dev 122(14): 1639-1649.

Lucanic, M., T. Garrett, I. Yu, F. Calahorro, A. Asadi Shahmirzadi, A. Miller, M. S. Gill, R. E. Hughes, L. Holden-Dye and G. J. Lithgow (2016). “Chemical activation of a food deprivation signal extends lifespan.” Aging Cell 15(5): 832-841.

Luo, J., S. H. Lee, L. VandeVrede, Z. Qin, M. Ben Aissa, J. Larson, A. F. Teich, O. Arancio, Y. D'Souza, A. Elharram, K. Koster, L. M. Tai, M. J. LaDu, B. M. Bennett and G. R. Thatcher (2016). “A multifunctional therapeutic approach to disease modification in multiple familial mouse models and a novel sporadic model of Alzheimer's disease.” Mol Neurodegener 11: 35.

Ma, L., Y. Jiang, Y. Dong, J. Gao, B. Du and D. Liu (2018). “Anti-TNF-alpha antibody attenuates subarachnoid hemorrhage-induced apoptosis in the hypothalamus by inhibiting the activation of Erk.” Neuropsychiatr Dis Treat 14: 525-536.

Mehta, D., R. Jackson, G. Paul, J. Shi and M. Sabbagh (2017). “Why do trials for Alzheimer's disease drugs keep failing? A discontinued drug perspective for 2010-2015.” Expert Opin Investig Drugs 26(6): 735-739.

Mobbs, C. V. and P. R. Hof (2007). Mechanisms of dietary restriction in aging and disease. Basel, Karger.

Pawluk, H., A. Wozniak, G. Grzesk, R. Kolodziejska, M. Kozakiewicz, E. Kopkowska, E. Grzechowiak and G. Kozera (2020). “The Role of Selected Pro-Inflammatory Cytokines in Pathogenesis of Ischemic Stroke.” Clin Intery Aging 15: 469-484.

Peters, K. R. (2013). “Utility of an effect size analysis for communicating treatment effectiveness: a case study of cholinesterase inhibitors for Alzheimer's disease.” J Am Geriatr Soc 61(7): 1170-1174.

Petrascheck, M., X. Ye and L. B. Buck (2007). “An antidepressant that extends lifespan in adult Caenorhabditis elegans.” Nature 450(7169): 553-556.

Raggi, A., D. Tasca, S. Panerai, W. Neri and R. Ferri (2015). “The burden of distress and related coping processes in family caregivers of patients with Alzheimer's disease living in the community.” J Neurol Sci 358(1-2): 77-81.

Spires-Jones, T. L., J. Attems and D. R. Thal (2017). “Interactions of pathological proteins in neuro degenerative diseases.” Acta Neuropathol.

Wang, X. P. and H. L. Ding (2008). “Alzheimer's disease: epidemiology, genetics, and beyond.” Neurosci Bull 24(2): 105-109.

Yang, H. M., S. Yang, S. S. Huang, B. S. Tang and J. F. Guo (2017). “Microglial Activation in the Pathogenesis of Huntington's Disease.” Front Aging Neurosci 9: 193.

Yang, X., Q. Zeng, M. Baris and G. Tezel (2020). “Transgenic inhibition of astroglial NF-kappaB restrains the neuroinflammatory and neurodegenerative outcomes of experimental mouse glaucoma.” J Neuroinflammation 17(1): 252.

Ye, X., J. M. Linton, N. J. Schork, L. B. Buck and M. Petrascheck (2014). “A pharmacological network for lifespan extension in Caenorhabditis elegans.” Aging Cell 13(2): 206-215.

Zhang, M., M. Poplawski, K. Yen, H. Cheng, E. Bloss, X. Zhu, H. Patel and C. V. Mobbs (2009). “Role of CBP and SATB-1 in aging, dietary restriction, and insulin-like signaling.” PLoS Biol 7(11): e1000245.

1. A compound according to Formula 1: 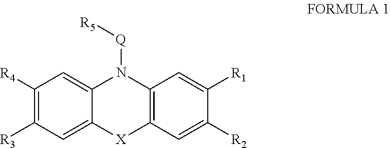
wherein:
X is selected from S or S═O;
Q is selected from CH₂ or CO;
R₁, R₂, R₃ and R₄, are selected from hydrogen, halogen, F, Br, Cl, CH₃, CF₃, OCH₃, COCH₃, CN, C(O)R⁸, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,
Wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; and
R₅ is selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl; and
pharmaceutically acceptable salts and enantiomers thereof.

2. A compound according to Formula 2: 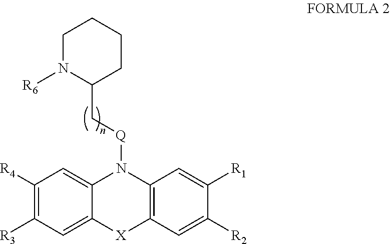
wherein;
X is selected from S or S═O;
Q is selected from CH₂ or CO;
n is selected from 0, 1, 2 or 3;
R₁, R₂, R₃ and R₄, are selected from hydrogen, halogen, F, Br, Cl, CH₃, CF₃, OCH₃, COCH₃, CN, C(O)R⁸ , optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl;
R⁶ is selected from hydrogen, CH₃, CF₃, OCH₃, COCH₃, CN, C(O)R⁸, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃ -C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,
Wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, and
pharmaceutically acceptable salts and enantiomers thereof.

3. A compound according to Formula 3: 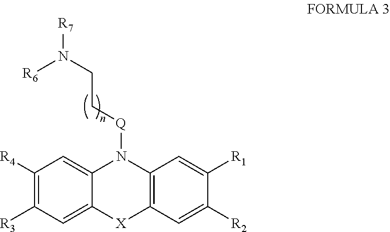
wherein;
X is selected from S or S═O;
Q is selected from CH₂ or CO;
n is selected from 0, 1, 2 or 3;
R₁, R₂, R₃ and R₄, are selected from hydrogen, halogen, F, Br, Cl, CH₃, CF₃, OCH₃, COCH₃, CN, C(O)R⁸ , optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl;
R₆ is selected from hydrogen, CH₃, CF₃, OCH₃, COCH₃, CN, C(O)R⁸ , optionally substituted C₁-C₈ alkyl, optionally substituted C₁-C₈alkoxyC₁-C₈alkyl, optionally substituted C₁-C₈ alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,
R₇ is selected from hydrogen, CH₃, CF₃, OCH₃, COCH₃, CN, C(O)R₈ , optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl,
wherein R₈ is optionally selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; and
pharmaceutically acceptable salts and enantiomers thereof.

4. A compound selected from the group consisting of:
TC₈₄-31, TC₈₄-37, TC₈₄-32, TC₈₄-38, TC₈₄-13, TC₈₄-20, TC₈₄-39, TC₈₄-110, TC₈₄-144, TC₈₄-125, TC₉₆-20, TC₈₄-124, TC₈₄-161, TC₈₄-162, TC₈₄-163, TC₉₆-32 and pharmaceutically acceptable salts thereof.

5. A compound according to claim 4, selected from the group consisting of:
a) 2-fluoro-10-(2-(1-methylpiperidin-2-yl)ethyl)-10H-phenothiazine(TC₈₄-110) and
b) 3-(2,3-dimethyl-10H-phenothiazin-10-yl)-N,N-dimethylpropan-1-amine (TC₈₄-124).

6. A method of treating a neurological disease,
comprising administering to a subject suffering from such disease a compound according to claim 4.

7. The method of claim 6 wherein the disease exhibits a neuroinflammatory component.

8. A method of treating COVID-19 and other coronaviral diseases, comprising administering to a subject suffering from such disease a compound according to claim 5.

9. A method of treating intracerebral hemorrhage, comprising administering to a subject suffering from intracerebral hemorrhage a compound according to claim 5.

10. A method of treating Alzheimer's or age-related and neurological diseases in a subject in need thereof, comprising:
administering to a subject having Alzheimer's or age related and neurological diseases, a compound according to claim 1.

11. A method of treating Alzheimer' or age-related disease in a subject in need thereof, comprising:
administering to a subject having Alzheimer's or age related disease, a pharmaceutical composition including a compound according to claim 1, and a pharmaceutically acceptable carrier.

12. A method of treating Alzheimer's or age-related and neurological diseases in a subject in need thereof, comprising:
administering to a subject having Alzheimer's or age related and neurological diseases, a compound according to claim 2.

13. A method of treating Alzheimer's or age-related and neurological diseases in a subject in need thereof, comprising:
administering to a subject having Alzheimer's or age related and neurological diseases, a compound according to claim 3.

14. A method of treating Alzheimer's or age-related disease in a subject in need thereof, comprising:
administering to a subject having Alzheimer's or age related disease, a pharmaceutical composition including a compound according to claim 1 and a pharmaceutically acceptable carrier.

15. A method of treating Alzheimer's or age-related disease in a subject in need thereof, comprising:
administering to a subject having Alzheimer's or age related disease, a pharmaceutical composition including a compound according to claim 2 and a pharmaceutically acceptable carrier.

16. A method of treating Alzheimer's or age-related disease in a subject in need thereof, comprising:
administering to a subject having Alzheimer's or age related disease, a pharmaceutical composition including a compound according to claim 3 and a pharmaceutically acceptable carrier.
